(19) United States
(12) Patent Application Publication
(10) Pub. No.: US 2022/0161251 A1
(43) Pub. Date: May 26, 2022

(54) CONTACTLESS DETECTION OF AN ABERRANT CONDITION

(71) Applicant: Singular Genomics Systems, Inc., La Jolla, CA (US)

(72) Inventors: Andrew Spaventa, La Jolla, CA (US); Christian Berrios, San Diego, CA (US); Eli N. Glezer, Del Mar, CA (US); Zachary Terranova, San Diego, CA (US)

(21) Appl. No.: 17/531,488

(22) Filed: Nov. 19, 2021

Related U.S. Application Data
(60) Provisional application No. 63/116,725, filed on Nov. 20, 2020.

(51) Int. Cl. B01L 3/00; G01N 33/483; G01N 33/487; G01N 33/53; B64C 39/02

(52) U.S. Cl. CPC B01L 3/50; G01N 33/4833; G01N 33/487; B64C 2201/128; B64C 39/024; B01L 2200/185; G01N 33/53

(57) ABSTRACT

Disclosed herein, inter alfa, are systems and methods for contactless evaluation of a biological sample.

### CROSS-REFERENCES TO RELATED APPLICATIONS

This application claims the benefit of U.S. Provisional Application No. 63/116,725, filed Nov. 20, 2020, which is incorporated herein by reference in its entirety and for all purposes.

### BACKGROUND

Patients showing symptoms of a disease or condition are often required to go to a medical office to receive a diagnosis and treatment. Infectious organisms are ubiquitous in a medical environment, despite vigorous efforts to maintain antisepsis. The presence of these organisms can result in further infection of other patients and medical personnel. The simple act of traveling to a medical office, such as a physician clinic, urgent care facility, or emergency room when infected with a communicable disease (e.g., influenza, tuberculosis, SARS-CoV-1, or SARS-CoV-2) potentially exposes hundreds if not thousands of individuals to the infection and risks the safety and wellbeing of others.

Transmission of an infectious disease occurs when an infectious organism moves from one host to another and causes disease in the new host. There are many factors that contribute to and influence transmission, however it is known that transmission can occur via contact, either direct contact with a fomite, or indirect contact with droplets or aerosols from expiratory releases (e.g., coughing, sneezing, or speaking). Limiting, or reducing, contact with the environment outside your own home is known to reduce the transmission and spread of contagions. Disclosed herein are solutions to these and other problems in the art.

### BRIEF SUMMARY

In an aspect is provided a method of detecting a disease in a subject, the method including: i) providing a collection device to a subject using an unmanned autonomous vehicle, ii) obtaining a sample from the subject and depositing the sample in the collection device; iii) delivering the collection device to an examination center; iv) identifying whether a disease is present in the sample by examining the sample, and v) detecting a disease in a subject when the presence of a disease is identified in the sample.

In an aspect is provided a method of diagnosing a subject with a disease, the method including: i) providing a collection device to a subject using an unmanned autonomous vehicle; ii) obtaining a sample from the subject and depositing the sample in the collection device; iii) delivering the collection device to an examination center; iv) identifying whether a disease is present in the sample by examining the sample, and v) diagnosing a subject with a disease when the presence of a disease is identified in the sample.

In an aspect is provided a method of detecting a pathogenic organism in a subject, the method including: i) providing a collection device to a subject using an unmanned autonomous vehicle; ii) obtaining a sample from the subject and depositing the sample in the collection device; iii) delivering the collection device to an examination center; iv) identifying whether a pathogenic organism is present in the sample by examining the sample, and v) detecting a pathogenic organism in a subject when the presence of a pathogenic organism is identified in the sample.

In an aspect is provided a method of detecting a disease in a subject, the method including: i) requesting transportation of an unmanned autonomous vehicle to a destination; ii) obtaining a sample from a subject and depositing the sample in a collection device; iii) providing the collection device to the unmanned autonomous vehicle; iv) delivering the collection device to an examination center; v) identifying whether a disease is present in the sample by examining the sample, and vi) detecting a disease in a subject when the presence of a disease is identified in the sample.

In an aspect is provided a method of evaluating a biological sample collected from a subject, the method including: i) requesting delivery of a collection device; ii) providing the collection device to the subject using an unmanned autonomous vehicle; iii) obtaining a biological sample from the subject and depositing the biological sample in the collection device; iv) delivering the collection device to an examination center; v) evaluating the biological sample by examining the biological sample.

In an aspect is provided a method of evaluating a biological sample collected from a subject, the method including: i) requesting transportation of an unmanned autonomous vehicle to a destination; ii) obtaining a sample from a subject and depositing the sample in a collection device; iii) providing the collection device to the unmanned autonomous vehicle; iv) delivering the collection device to an examination center; v) evaluating the biological sample by examining the biological sample.

In an aspect is provided a method of providing a collection device to a subject, the method including: i) receiving from a subject a request a request for delivery of one or more collection devices; ii) providing one or more collection devices to the subject using an unmanned autonomous vehicle, wherein the one or more collection devices include a biological collection device.

In an aspect is provided a kit including a collection device.

In an aspect is provided a computing device configured to enable the evaluation of a biological sample collected from a subject, including: a processor; memory in electronic communication with the processor; and instructions stored in the memory, the instructions being executable by the processor to: a. request for delivery of a collection device; b. communicate with a server to provide the collection device using an unmanned autonomous vehicle; c. communicate with a server to deliver the collection device containing a biological sample to an examination center; d. receive data associated with the evaluation of the biological sample from the examination center.

In an aspect is provided a system, including: at least one data processor; and at least one memory storing instructions, which when executed by the at least one data processor, result in operations including: receiving, from a client device, a request for a collection device;

responding to the request by at least identifying a first unmanned autonomous vehicle for delivering the collection device, the first unmanned autonomous vehicle being identified based at least on a first location associated with the request and/or a second location of the first unmanned autonomous vehicle; sending, to the first unmanned autonomous vehicle, a first command to deliver the collection device to the first location; and sending, to the first unmanned autonomous vehicle or a second unmanned autonomous vehicle, a second command to deliver the collection device including a sample from the first location to an examination center.

In an aspect is provided an unmanned autonomous vehicle, including: at least one data processor; and at least one memory storing instructions, which when executed by the at least one data processor, result in operations including: sending a first message including a current location of the unmanned autonomous vehicle and a current capacity of the unmanned autonomous vehicle; receiving a command to deliver a collection device to a location or to deliver the collection device from the location to an examination center; and in response to detecting a deposit and/or a removal of the collection device at the location, sending a second message including an updated location of the unmanned autonomous vehicle and/or an updated capacity of the unmanned autonomous vehicle.

In an aspect is provided a system, including: an unmanned autonomous vehicles; and a central server communicatively coupled with the unmanned autonomous vehicle, the central server including at least one data processor and at least one memory, the at least one memory may store instructions, which when executed by the at least one data processor, result in operations including: receiving, from a client device, a request for a collection device; receiving, from the unmanned autonomous vehicle, a message including a current location of the unmanned autonomous vehicle; responding to the request by at least identifying the unmanned autonomous vehicle for delivering the collection device, the unmanned autonomous vehicle being identified based at least on a location associated with the request and the current location of the unmanned autonomous vehicle; and in response to identifying the unmanned autonomous vehicle for delivering the collection device, sending, to the unmanned autonomous vehicle, a command to deliver the collection device to the location associated with the request.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of an unmanned autonomous vehicle biological specimen evaluation system, in accordance with some example embodiments;

FIG. 2 depicts a flowchart illustrating an example of a process for evaluating a biological sample, in accordance with some example embodiments; and

FIG. 3 depicts a block diagram illustrating an example of a computing system, in accordance with some example embodiments.

When practical, similar reference numbers denote similar structures, features, or elements.

### DETAILED DESCRIPTION

The aspects and embodiments described herein relate to contactless methods of evaluating biological specimens utilizing unmanned autonomous vehicles.

I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, N.Y. 1994); and Sambrook and Green, Molecular Cloning: A Laboratory Manual, 4th Edition (2012). Methods, devices and materials similar or equivalent to those described herein can be used in the practice of this invention.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of. ” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances, two or more associated species are “tethered”, “coated”, “attached”, or “immobilized” to one another or to a common solid or semisolid support. An association may refer to covalent or non-covalent means for attaching labels to solid or semi-solid supports such as beads. In embodiments, primers on or bound to a solid support are covalently attached to the solid support. An association may comprise hybridization between a target and a label.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. Contacting, a used herein, may refer to a human physically touching a biological sample. In the alternative, “contactless” refers to a means of not allowing two distinct species to become sufficiently proximal to react, interact or physically touch.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides.

As used herein, the terms “polynucleotide primer” and “primer” refers to any polynucleotide molecule that may hybridize to a polynucleotide template, be bound by a polymerase, and be extended in a template-directed process for nucleic acid synthesis. Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

Typically, a probe oligonucleotide is complementary to a target polynucleotide or portion thereof, and further comprises a label (such as a binding moiety) or is attached to a surface, such that hybridization to the probe oligonucleotide permits the selective isolation of probe-bound polynucleotides from unbound polynucleotides in a population. A probe oligonucleotide may or may not also be used as a primer. In embodiments, examining the sample include contacting the sample with a probe oligonucleotide and detecting the probe oligonucleotide.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

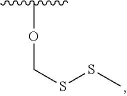 [SMILES: *OCSSC]

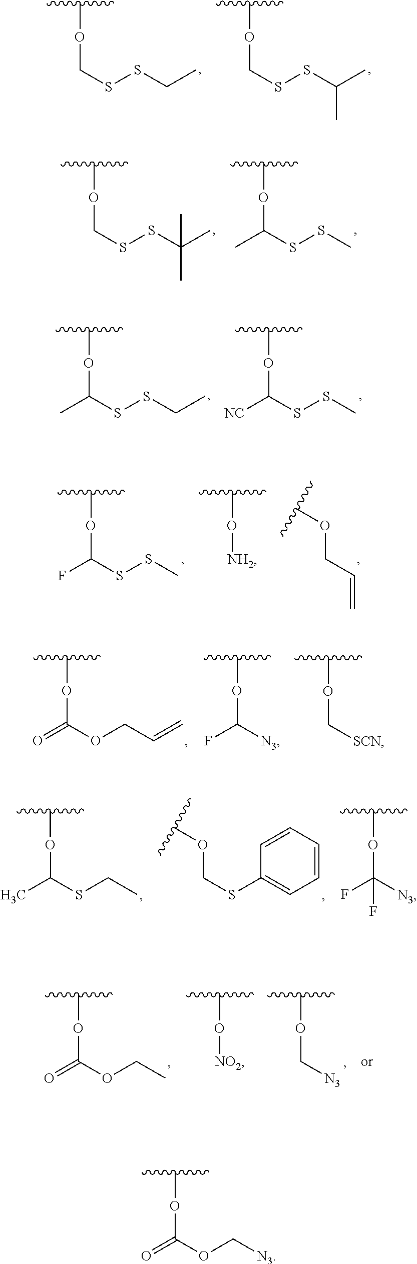

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the terms “reversible blocking groups” and “reversible terminators” are used in accordance with their plain and ordinary meanings and refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH2 reversible terminator, a 3′-O-allyl reversible terminator, or a 3′-O-azidomethyl reversible terminator.

As used herein, the term “barcode” or “index” or “unique molecular identifier (UMI)” refers to a known nucleic acid sequence that allows some feature with which the barcode is associated to be identified. Typically, a barcode is unique to a particular feature in a pool of barcodes that differ from one another in sequence, and each of which is associated with a different feature. In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are 10-50 nucleotides in length, such as 15-40 or 20-30 nucleotides in length. In a pool of different barcodes, barcodes may have the same or different lengths. In general, barcodes are of sufficient length and comprise sequences that are sufficiently different to allow the identification of associated features (e.g., a binding moiety or analyte) based on barcodes with which they are associated. In embodiments, a barcode can be identified accurately after the mutation, insertion, or deletion of one or more nucleotides in the barcode sequence, such as the mutation, insertion, or deletion of 1, 2, 3, 4, 5, or more nucleotides. In embodiments, each barcode in a plurality of barcodes differs from every other barcode in the plurality by at least three nucleotide positions, such as at least 3, 4, 5, 6, 7, 8, 9, 10, or more nucleotide positions.

As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide comprises a label (such as a dye). In embodiments, the label is not associated with any particular nucleotide, but detection of the label identifies whether one or more nucleotides having a known identity were added during an extension step (such as in the case of pyrosequencing).

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044).

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In embodiments, the target polynucleotide is a polynucleotide from a sample. The target sequence may be a portion of a gene, a regulatory sequence, genomic DNA, cDNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. In the context of selective sequencing, “target polynucleotide(s)” refers to the subset of polynucleotide(s) to be sequenced from within a starting population of polynucleotides.

As used herein, the terms “specific”, “specifically”, “specificity”, or the like of a compound refers to the compound's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the term “sequencing reaction mixture” is used in accordance with its plain and ordinary meaning and refers to an aqueous mixture that contains the reagents necessary to allow a nucleotide or nucleotide analogue to be added to a DNA strand by a DNA polymerase.

As used herein, the term “extension” or “elongation” is used in accordance with their plain and ordinary meanings and refer to synthesis by a polymerase of a new polynucleotide strand complementary to a template strand by adding free nucleotides from a reaction mixture that are complementary to the template in a 5′-to-3′ direction, including condensing a 5′-phosphate group of a dNTPs with a 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction comprises a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that comprises a polymerase chain reaction (PCR). Conditions conducive to amplification (i.e., amplification conditions) are well known and often comprise at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).A nucleic acid can be amplified by a thermocycling method or by an isothermal amplification method. In some embodiments, a rolling circle amplification method is used. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments solid phase amplification comprises a nucleic acid amplification reaction comprising only one species of oligonucleotide primer immobilized to a surface or substrate. In certain embodiments solid phase amplification comprises a plurality of different immobilized oligonucleotide primer species. In some embodiments solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., US patent publication US2013/0012399), the like or combinations thereof.

Provided herein are methods and compositions for examining or analyzing a sample (e.g., a sample from a subject, alternatively referred to herein as a specimen or a biological sample). A sample can be provided from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen (e.g., a biological specimen) that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may comprise cells or tissues that are normal, healthy, diseased (e.g., infected), and/or cancerous (e.g., cancer cells). A sample obtained from a subject may comprise cells or cellular material (e.g., nucleic acids) of multiple organisms (e.g., virus nucleic acid, fetal nucleic acid, bacterial nucleic acid, parasite nucleic acid). A sample may be collected from a living or dead subject. A sample may be collected fresh from a subject or may have undergone some form of pre-processing, storage, or transport.

In some embodiments, a sample includes a nucleic acid, or fragments thereof. A sample can comprise nucleic acids obtained from one or more subjects. In some embodiments a sample comprises nucleic acid obtained from a single subject. In some embodiments, a sample comprises a mixture of nucleic acids. A mixture of nucleic acids can comprise two or more nucleic acid species having different nucleotide sequences, different fragment lengths, different origins (e.g., genomic origins, cell or tissue origins, subject origins, the like or combinations thereof), or combinations thereof. A sample may comprise synthetic nucleic acid. In some embodiments, a sample includes a biomarker, or fragments thereof.

One or more, two or more, three or more, four or more, five or more, six or more, seven or more, eight or more, ten or more, twelve or more, fifteen or more, or twenty or more different types of samples may be collected from a subject. A single type of sample or a plurality of types of samples may be collected from the subject simultaneously or at different times. A single type of sample or a plurality of types of samples may be received or capable of being received by the device simultaneously or at different times. A plurality of types of samples may be processed by the device in parallel and/or in sequence. For example, a device may be capable of receiving both a bodily fluid and a tissue, or a stool sample and a bodily fluid. In another example, a device may be capable of receiving a plurality of types of bodily fluids, such as blood and urine. For example, the device may be capable of receiving one or more type, two or more type, three or more types, four or more types, five or more types, six or more types, seven or more types, eight or more types, ten or more types, or twenty or more types of bodily fluid.

Any volume of sample may be provided from the subject. Examples of volumes may include, but are not limited to, about 10 mL or less, 5 mL or less, 3 mL or less, 1 microliter (μL, also “uL” herein) or less, 500 μL, or less, 300 μL, or less, 250 μL, or less, 200 μL, or less, 170 μL, or less, 150 μL, or less, 125 μL, or less, 100 μL, or less, 75 μL, or less, 50 μL, or less, 25 μL, or less, 20 μL, or less, 15 μL, or less, 10 μL, or less, 5 μL, or less, 3 μL, or less, 1 μL, or less, 500 nL or less, 250 nL or less, 100 nL or less, 50 nL or less, 20 nL or less, 10 nL or less, 5 nL or less, 1 nL or less, 500 pL or less, 100 pL or less, 50 pL or less, or 1 pL or less. The amount of sample may be about a drop of a sample. The amount of sample may be the amount collected from a pricked finger, swab, sponge, or paper. The amount of sample may be the amount collected from a microneedle. Any volume, including those described herein, may be provided to the collection.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation (e.g., an oncogene). A subject may be undergoing screening, diagnosis, treatment, monitoring and/or disease prevention. The subject may or may not be under the care of a health care professional.

The term “analyte” or “biomarker” and its plural and other forms, as used herein, includes without limitation drugs, prodrugs, pharmaceutical agents, drug metabolites, biomarkers such as expressed proteins and cell markers, antibodies, antigens, proteins, hormones, polypeptides, glycoproteins, polysaccharides, lipids, viruses, cholesterol, polysaccharides, nucleic acids, genes, nucleic acids, and combinations thereof.

As used herein, the term “disease state” is used in accordance with its plain and ordinary meaning and refers to any abnormal biological or aberrant condition of a cell, tissue, or organism. The presence of a disease state may be identified by the same collection of biological constituents (e.g., biomarkers) used to determine the cell's biological state. In general, a disease state will be detrimental to a biological system (e.g., a cell or organism). A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level or severity (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). In embodiments, methods provided herein are also applicable to monitoring the disease state or states of a subject undergoing one or more therapies. Thus, the present disclosure also provides, in some embodiments, methods for determining or monitoring efficacy of a therapy or therapies (i.e., determining a level of therapeutic effect) upon a subject. In embodiments, methods of the present disclosure can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, e.g., a particular disease state or therapy, even at a stage where changes in protein activity are undetectable.

As used herein, the term “cell count” refers to a qualitative or quantitative assessment of the number of cells in a sample, or in a volume, or in a field of view, or on a surface. An estimate of the number of cells in a sample, e.g., by absorbance of light passing through, or scattered or otherwise altered by cells in a sample; by light or other radiation emitted by cells expressing fluorescent or otherwise detectable proteins, or labeled by dyes, radionuclides, or other markers; an enumeration of the number of cells in an image (e.g., an image of a blood or urine sample, a tissue slice, or other biological sample, acquired by a camera, a microscope, or other optical imaging device), and a quantitative measurement of the number of cells adherent to a surface are all examples of a cell count.

As used herein, the term “cytometry” refers to observations, analysis, methods, and results regarding cells of a biological sample, where the cells are substantially at rest in a fluid or on a substrate. Cells detected and analyzed by cytometry may be detected and measured by any optical, electrical or acoustic detector. Cytometry may include preparing and analyzing images of cells in or from a biological sample (e.g., two-dimensional images). The cells may be labeled (e.g., with fluorescent, chemiluminescent, enzymatic, or other labels) and plated (e.g., allowed to settle on a substrate) and detected. Cells may be, for example, imaged by a camera. A microscope may be used for cell imaging in cytometry; for example, cells may be imaged by a camera and a microscope, e.g., by a camera forming an image using a microscope. An image formed by, and used for, cytometry typically includes more than one cell. As used herein, the term “cytometry” refers to measurements made without substantial cell motion with respect to the device (e.g., the cells are observed and imaged while at rest with respect to a surface of the device). In this regard, the term “cytometry” as used herein is different from, and refers to different observations, analysis, methods, and results than are referred to by the more limited term “flow cytometry.”

As used herein, the term “flow cytometry” refers to observations and analysis of cells, where the cells are in motion within a device, or with respect to a surface of a device. Flow cytometry typically uses a mobile liquid medium that sequentially carries individual cells to an optical, electrical or acoustic detector; e.g., in a typical flow cytometer, cells are carried by a moving fluid past a stationary detector.

As used herein, the term “microscopy” refers to the use of magnifying lenses and other optical methods to provide an image of a target, such as a cell or a plurality of cells. Microscopy typically uses optical or acoustic means to detect stationary cells, generally by recording at least one magnified image. An image formed by microscopy typically provides greater resolution than is possible when viewing the target by eye alone; for example, the image may be enlarged.

Optical methods and techniques used in microscopy, in addition to the use of lenses for refraction, include the use of mirrors, prisms, filters, polarizers, gratings, grids, light sources, scanning methods and techniques (e.g., as used in confocal microscopy), special apertures (e.g., a pin-hole), and other methods.

As used herein, “spectroscopy” refers to assays and measurements using light intensity, including light intensity as a function of light wavelength to detect and assess a sample. Spectroscopy includes measurements of the reflection or transmission of electromagnetic waves, including visible, UV, and infrared light. Spectroscopy includes any and all assays that produce luminescence or change light (e.g., color chemistry). These may include one or more of the following: spectrophotometry, fluorimetry, luminometry, turbidimetry, nephelometry, refractometry, polarimetry, and measurement of agglutination.

The term “fluorimetry” as used herein refers to measuring the light emitted by a fluorescent molecule coupled to a subject upon exciting the fluorescent molecule with incident light.

As used herein, “luminometry” refers to measurements and observations that use no external illumination method, but instead detects electromagnetic radiation emitted by or from the object, chemical reaction, or area of interest. The emitted light may be weak, and thus luminometry may require the detection of low light or other radiation levels; such signals can be detected using an extremely sensitive sensor such as a photomultiplier tube (PMT). Luminometry includes assays that produce chemiluminescence, such as those using luciferases or some assays using peroxidases.

As used herein, “turbidimetry” refers to detection, measurement, or observation of a sample or reaction in a sample by backlighting the sample and components within the sample with white light, with the result being sensed by an imaging sensor. The reduction of the intensity of the transmitted light is measured (the intensity of the incident light being known). Turbidimetry may be used, for example, to determine a concentration of cells in solution. In some embodiments, turbidimetry is measured by nephelometry.

As used herein, the term “nephelometry” refers to measurements of light that is transmitted or scattered after passing through a suspension, e.g., a suspension of target analytes in a solution. For example, the amount of a substrate bound to an immunoglobin such as IgM, IgG, and IgA may be measured by nephelometry.

As used herein, the term “polarimetry” refers to measurements of the polarization of light or other electromagnetic radiation following reflection, refraction, or other contact with subject object or field. Polarimetry assays include circular dichroism, which may provide structural information and light scattering assays, which may provide information about the size and/or shape of the subject. One nonlimiting example of light scattering assays uses dynamic light scattering (DLS).

As used herein, the terms “colorant” and “chromogen” refer to a compound which produces or provides a detectable change in color, absorbance, turbidity, or other optical property of a medium. Chromagens may be used to signal the occurrence, progress, or results of a chemical reaction, which may be detected and measured by colorimetric or other means (e.g., by luminometer, spectrophotometer, or other light detector).

As used herein, the term “colored product,” is used to refer to the products that result from addition of a colorant to a solution. For example, addition of a colorant to a solution may result in a reaction effective to alter an optical property of the solution. Such a reaction may result in the formation of molecules originally not present in the solution, or may result in the aggregation of molecules or compounds previously in the solution, or may result in the degradation or other alteration of molecules or compounds previously in the solution, effective to alter the color, absorbance, and/or other optical properties of a solution to which a colorant is added.

As used herein, the terms “reflex” and “reflex testing” refer to the initiation, modification or repetition of a protocol, measurement, assay method, or analysis based on information or results obtained following an initial measurement, assay, or analysis. A reflex test may be performed where information is obtained by an initial measurement that can be supplemented by further testing, suggests that a more precise or specific test should be performed, or that an additional test should be performed for an analyte or condition related to or suggested by the initial measurement. Typically, a reflex test is performed based on the results of an initial test; the initial test is typically less sensitive, cheaper or faster than the reflex test. For example, reflex testing may occur when, after detecting a possible abnormality with an initial PAP smear, a more specific or precise test, such as one using nucleic acid analysis, may be performed to more accurately assess the subject's condition. Another example of reflex testing might include measurement of the folate level in the blood of a subject based on finding a low hematocrit level in an initial blood test.

As used herein, the term “receptor-based assay” refers to an assay which utilizes, or detects, the binding of a receptor to its ligand, or the dissociation of a ligand from its receptor. Such assays may use the binding directly to detect or quantify the presence or amount of a receptor or ligand, may use competitive binding techniques to detect or quantify the presence or amount of a target molecule in a sample, or may detect or quantify the presence or amount of a target by use of other methods based on binding between a receptor and ligand or ligands.

As used herein, the term “immunoassay” refers to tests which utilize antibodies (including antibody fragments) and their binding to target molecules to label, identify, quantify, or otherwise provide information regarding the presence, amounts, and properties of target molecules and samples containing them. One useful immunoassay that can be run on a device disclosed herein is an ELISA (Enzyme-Linked ImmunoSorbent Assay) Immunoassays also include, for example, competitive binding assays, sandwich assays, Western blots, and other assays utilizing antibodies and antibody fragments.

As used herein, the term “enzymatic assay” refers to an assay which utilizes, or detects, the presence or action of an enzyme. For example, an assay which provides a substrate for a target enzyme, and detects the presence of that enzyme, or quantifies the activity of that enzyme in a sample following addition of the substrate is an enzymatic assay. An assay which utilizes the enzymatic production of a detectable substance is another example of an enzymatic assay; for example, colorimetric assays (e.g., in which a detectable product is produced by an enzyme, which may be an endogenous enzyme or which may be supplied with the assay reagents) such as assays in which horseradish peroxidase or alkaline phosphatase is used to produce a colored product as an indicator of the progress of the reaction or presence of a target, are enzymatic assays.

As used herein, “Clinical Laboratory Improvement Amendments” and “CLIA” refer to sections of 42 U.S.C. Part F, e.g., subpart 2, sections 263a through 263a7, Federal Regulations 42 CFR Chapter IV (sections 493.1 to 493.2001), and related laws, regulations, and as amended. Regulations pursuant to CLIA are administered by the Centers for Medicare and Medicaid Services (CMS) of the United States Department of Health and Human Services.

As used herein, the term “CLIA-compliant” means that a device, a procedure, an operation, a laboratory, or other facility, complies with CLIA. As used herein, the term “CLIA-certified” means a device, a procedure, an operation, a laboratory, or other facility, that has been certified, by an appropriate regulatory body empowered to do so, as compliant with CLIA. As used herein, the term “CLIA-compliant laboratory” means a laboratory, or other facility, that complies with CLIA. As used herein, the term “CLIA-certified laboratory” means a laboratory, or other facility, that has been certified, by an appropriate regulatory body empowered to do so, as being compliant with CLIA. A CLIA-certified laboratory is a CLIA-compliant laboratory. As used herein, a device that is a “CLIA-compliant device” is a device that complies with CLIA or whose use complies with CLIA. As used herein, a “CLIA-waived device” is a device for which a certificate of waiver, under CLIA, has been issued by an appropriate regulatory body empowered to do so, as compliant with CLIA or whose use complies with a certificate of waiver, under CLIA, issued by an appropriate regulatory body empowered to do so. A CLIA-waived device is a CLIA-compliant device. As used herein, a “CLIA-certified device” is a device that has been certified, by an appropriate regulatory body empowered to do so, as compliant with CLIA or whose use complies with a certification issued under CLIA by an appropriate regulatory body empowered to do so. A CLIA-certified device is a CLIA-compliant device.

Any description herein of a laboratory may apply to an authorized analytical facility and vice versa. In some instances, the laboratory may be certified by a governmental agency or professional association. A laboratory may receive certification or oversight by a regulatory body. In one example, the laboratory may be certified by an entity, such as Centers for Medicare & Medicaid Services (CMS), College of American Pathologists, ISO standards 15189 or 17025 or equivalents thereof. For instance, an authorized analytical facility may be a Clinical Laboratory Improvement Amendments (CLIA) certified laboratory in the United States or its equivalent in a foreign jurisdiction.

An authorized analytical facility is typically subject to oversight or regulation. For example, a laboratory may have oversight by a board-certified entity (which may include one or more board-certified personnel). In some embodiments, oversight can include validating one or more clinical test. Oversight may also include assessing the performance of, correcting, calibrating, running controls, replicates, adjusting, or analyzing one or more clinical test. Oversight can include evaluation of one or more sets of data to provide a quality control for a clinical test. The authorized analytical facility can have one or more qualified person providing the oversight. For example, one or more pathologist or other health care professional may review data and/or analysis that is processed by the facility. At an authorized analytical facility, a trained pathologist or other certified health care professional may provide oversight. In some instances, the certified health care professional providing oversight may be one or more of the following: a doctor certified in pathology, a doctor with laboratory training or experience in the specialty areas of service for which the health care professional is responsible, or an individual with experience or laboratory training in the specialty.

A health care professional may include a person or entity that is associated with the health care system. A health care professional may be a medical health care provider. A health care professional may be a doctor. A health care professional may be an individual or an institution that provides preventive, curative, promotional or rehabilitative health care services in a systematic way to individuals, families and/or communities. Examples of health care professionals may include physicians (including general practitioners and specialists), dentists, audiologists, speech pathologists, physician assistants, nurses, midwives, pharmacologists, pharmacists, dietitians, therapists, psychologists, chiropractors, clinical officers, physical therapists, phlebotomists, occupational therapists, optometrists, emergency medical technicians, paramedics, medical laboratory technicians, medical prosthetic technicians, radiographers, social workers, and a wide variety of other human resources trained to provide some type of health care service. A health care professional may or may not be certified to write prescriptions. A health care professional may work in or be affiliated with hospitals, health care centers and other service delivery points, or also in academic training, research and administration. Some health care professionals may provide care and treatment services for patients in private homes. Community health workers may work outside of formal health care institutions. Managers of health care services, medical records and health information technicians and other support workers may also be health care professionals or affiliated with a health care provider.

In some embodiments, the health care professional may already be familiar with the subject or have communicated with the subject. The subject may be a patient of the health care professional. In some instances, the health care professional may have prescribed the subject to undergo a clinical test. The health care professional may have instructed or suggested to the subject to undergo a clinical test conducted at the sample collection site or by the laboratory. In one example, the health care professional may be the subject's primary care physician. The health care professional may be any type of physician for the subject (including general practitioners, and specialists).

A health care professional may receive a report from an authorized analytical facility. The health care professional that receives a report may be an ordering health care professional or health care professional in the analytical facility and/or sample collection site. The report may contain the results of the examination or analysis of the sample.

The term “unmanned autonomous vehicle” (or “UAV”) is used herein to refer to one of various types of autonomous vehicles (e.g., autonomous aircraft, land vehicles, waterborne vehicles, or a combination thereof) that may not utilize onboard, human pilots. A UAV may include an onboard computing device configured to operate the UAV without remote operating instructions (i.e., autonomously), such as from a human operator or remote computing device. Alternatively, the onboard computing device may be configured to operate the UAV with remote operating instruction or updates to instructions stored in a memory of the onboard computing device. The UAV may be propelled for movement in any of a number of known ways. For example, a plurality of propulsion units, each including one or more propellers or jets, may provide propulsion or lifting forces for the UAV and any payload (e.g., item(s) for delivery) carried by the UAV for travel or movement. In addition, or alternatively, the UAV may include wheels, tank-tread, floatation devices, or other non-aerial movement mechanisms to enable movement on the ground, across water, or under water. Various embodiments are described with reference to a UAV, particularly an aerial UAV, for ease of reference. However, the description of any particular UAV is not intended to limit the scope of the claims to unmanned aerial vehicles. Further, the UAV may be powered by one or more types of power source, such as electrical, chemical, electro-chemical, or other power reserve, which may power the propulsion units, the onboard computing device, and/or other onboard components.

As used herein, the terms “request for delivery”, “request for a collection device”, “request for delivery of a collection device”, and the like refer to an electronic request or agreement placed for at least one collection device, including sufficient details to arrange delivery of the at least one collection device.

A collection device may be any suitable vessel, container, or material, such as a microfluidic paper-based analytical device (μPAD), cotton swab, transdermal patch, or device configured to collect and/or store a bodily fluid or biological sample that may be delivered by a UAV. In some embodiments, the collection device includes a nasal swab, nasopharyngeal swab, oropharyngeal swab, throat swab, buccal swab, oral fluid swab, stool swab, tonsil swab, vaginal swab, cervical swab, blood swab, or wound swab that may be delivered by a UAV. In some embodiments, the collection device includes a swab, at least one vessel, a buffer, or preservative that may be delivered by a UAV. A request for delivery may include a transaction description, as well as a description of the collection device, the quantities requested, item origin and delivery locations, required lead time for delivery, and the terms of agreement included as part of the request for delivery. In embodiments, a collection device includes the instrument useful for obtaining the sample and one or more containers to store the instrument.

As used herein, the term “request parameters” or “command to deliver” refers to information that may pertain to a request for delivery of a collection device and/or biological sample, including collection device(s) and/or biological sample(s) to be delivered, a destination for delivery, delivery or mission requirements, etc. Request parameters may include information obtained directly and indirectly from the request for delivery. For example, mission parameters may include information for effecting delivery of the collection device and/or biological sample, which may be determined indirectly from the request for delivery. Such mission parameters are included within the term request parameters. Thus, request parameters may include information selected from a group including a timing of delivery, when and/or where the request for delivery was placed, when delivery of the collection device and/or biological sample is expected, when delivery of the collection device and/or biological sample is guaranteed, one or more locations of delivery, a travel route for delivery, current weather conditions, predicted weather conditions, travel time, a perishability of the collection device and/or biological sample, a required collection device and/or biological sample protection, one or more types of packaging (required or desired), a desired collection device and/or biological sample protection, a monetary value of the collection device and/or biological sample, a cost to deliver the collection device and/or biological sample, and other contextual information that may affect the delivery of a collection device and/or biological sample in the request for delivery. The cost to deliver the collection device and/or biological sample may take into account an opportunity cost of delivering the collection device and/or biological sample now versus waiting for a future order (e.g., that might have priority or be combined with another order to a nearby location or risk delivering in current weather or time of day). In addition, request parameters may include (but is not limited to) mission information identifying more than one destination, a route to each destination, map or travel directions, directions of travel, how the UAV will travel (e.g., air, land, and/or sea), chances of success and/or failure, timing information relating to each destination, and the like. In addition, request parameters may include route details, distance, travel speed(s), flight restrictions along a route, obstacles, permissions, or other information that may be useful for traveling, landing, fueling, recharging, and assisting the UAV in reaching a destination. Further, the request parameters may include delivery information, such as hazards to avoid, details regarding how delivery should be made (e.g., drop from height, land and wait, land and release, etc.), and/or proper delivery verification information. Proper delivery verification information may include security information and/or enable recognition of a drop zone or other visual recognition information (e.g., facial recognition corresponding to a recipient or landmark/signage recognition). In embodiments, the trajectory of the UAV is generated given the start and end locations included in the request parameters. In embodiments, the UAV navigates to a given location using a global positioning system (GPS) and onboard lidar/visual/inertial sensors. In embodiments, the UAV periodically (e.g., 1-10 Hz) reports its position and/or orientation to a central server for automated travel path monitoring. In embodiments, the UAV verifies the pick-up location by visually mapping the observed terrain and comparing to a known map. In embodiments, recognition of the drop zone includes determining that an area is without hazards (e.g., people, trees, vehicles, homes, etc.).

The term “computing device” is used herein to refer to an electronic device equipped with at least a processor. Examples of computing devices may include UAV travel control and/or mission management computers, mobile devices (e.g., cellular telephones, wearable devices, smartphones, smartwatches, web-pads, tablet computers, Internet enabled cellular telephones, Wi-Fi® enabled electronic devices, personal data assistants (PDAs), laptop computers, etc.), personal computers, and server computing devices. In various embodiments, computing devices may be configured with memory and/or storage as well as networking capabilities, such as network transceiver(s) and antenna(s) configured to establish a wide area network (WAN) connection (e.g., a cellular network connection, etc.) and/or a local area network (LAN) connection (e.g., a wired/wireless connection to the Internet via a Wi-Fi® router, etc.). In embodiments, the computing device is a mobile device, such as a cellular telephone, wearable device, or smartphone (e.g., iPhone, Android, Blackberry, Palm, Symbian, or Windows).

The term “server” as used herein refers to any computing device capable of functioning as a server, such as a master exchange server, web server, and a personal or mobile computing device configured with software to execute server functions (e.g., a “light server”). Thus, various computing devices may function as a server, such as any one or all of cellular telephones, smart-phones, web-pads, tablet computers, Internet enabled cellular telephones, Wi-Fi® enabled electronic devices, laptop computers, personal computers, and similar electronic devices equipped with at least a processor, memory, and configured to receive electronic orders and communicate with a UAV. A server may be a dedicated computing device or a computing device including a server module (e.g., running an application that may cause the computing device to operate as a server). A server module (or server application) may be a full function server module, or a light or secondary server module (e.g., light or secondary server application). A light server or secondary server may be a slimmed-down version of server type functionality that may be implemented on a personal or mobile computing device thereby enabling the light or secondary server to function as an Internet server (e.g., an enterprise e-mail server) to a limited extent, such as necessary to provide the functionality described herein.

As used in this application, the terms “component”, “module”, “system”, and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

As used in this application, the term “device ID” and the like are intended to refer to unique computer and network characteristics may include IP address, MAC address, serial number of CPU, serial number of hard drive, serial number of software loaded on the computer, cadence of user typing, characteristics of the data input devices such as web-cams, or mechanisms already used to uniquely identify computers and/or network connections. Such characteristics may be used to identify a client device used by a user during a verification session with a verification host. The identity of the client device may be another factor used in assessing a confidence level concerning the user's identity or possession of specified personal attributes.

As used in this application, the terms “identity information”, “authenticating information”, “identity of a client” , “identity of a subject” and the like are intended to refer to information used for proving a user identity, which includes at least one of a user account, a user password, user certificate information, and user biological feature information. The user biological feature information includes face feature information, fingerprint feature information, iris feature information, or a palm geometrical shape. The certificate information includes a certificate number, a name, the date of birth, an issuing authority, and a validity period. A certificate may be an identity card, a driving license, a social security card, a passport, and the like. Collecting the user identity information may be specifically acquiring the user identity information input by a user. For example, a character string entered in a user account input box is acquired as a user account, and a character string entered in a user password input box is acquired as a user password. For another example, certificate information input in a certificate information input box is acquired. Collecting the user identity information may also be acquiring the user identity information by calling a camera, a sensor, and the like. For example, a certificate image or a face image is acquired by camera scanning, and the fingerprint feature information, the iris feature information, and the like are obtained by sensor scanning.

A laboratory, examination center, device, or other entity or software may perform analysis on the data in real-time. Analysis may include qualitative and/or quantitative evaluation of a sample. A laboratory, examination center, device, or other entity may analyze and or report the data within 48 hours or less, 36 hours or less, 24 hours or less, 12 hours or less, 8 hours or less, 6 hours or less, 4 hours or less, 3 hours or less, 2 hours or less, 1 hour or less, 45 minutes or less, 30 minutes or less, 20 minutes or less, 15 minutes or less, 10 minutes or less, 5 minutes or less, 3 minutes or less, 1 minute or less, 30 seconds or less, 15 seconds or less, 10 seconds or less, 5 seconds or less, or 1 second or less. The analysis may include the comparison of the data with one or more threshold value. The analysis may or may not include review by a pathologist or other qualified person. The time included for analysis may or may not include time to generate a report based on the data. The time included for analysis may or may not include the time it takes to transmit a report to a health care professional.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the claims.

The hardware and systems used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of receiver smart objects, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In embodiments, the functions of the systems described may be implemented in hardware, software, firmware, or any combination thereof. If implemented in software, the functions may be stored as one or more instructions or code on a non-transitory computer-readable storage medium or non-transitory processor-readable storage medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include RAM, ROM, EEPROM, FLASH memory, CD-ROM or other optical disk storage, magnetic disk storage or other magnetic storage smart objects, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

II. Methods

In an aspect is provided a method of detecting a disease or disorder in a subject. In embodiments, the method includes i) providing a collection device to a subject using an unmanned autonomous vehicle, ii) obtaining a sample from the subject and depositing the sample in the collection device; iii) delivering the collection device to an examination center; iv) identifying whether a disease or disorder is present in the sample by examining the sample, and v) detecting a disease or disorder in a subject when the presence of a disease is identified in the sample. In embodiments, the method includes i) providing an unmanned autonomous vehicle, ii) obtaining a sample from the subject and depositing the sample in a collection device; iii) delivering the collection device to an examination center; iv) identifying whether a disease is present in the sample by examining the sample, and v) detecting a disease in a subject when the presence of a disease is identified in the sample. In embodiments, the subject uses a collection device (e.g., a collection device the subject purchased from a store, pharmacy, or medical office). In embodiments, the unmanned autonomous vehicle may be requested by the subject. In embodiments, the unmanned autonomous vehicle is provided by a third party (e.g., a physician, or other health care professional) requesting delivery to a subject.

In embodiments, the presence of a disease (i,e., a disease state) is identified when one or more biomarkers or agents associated with the disease are detected (e.g., detected at a sufficient level or threshold, for example, relative to a control). In embodiments, the presence of the disease is identified in a sample from a subject. In embodiments, the disease is detected in the sample by examination of the sample, for example, wherein the examination includes identification of one or more biomarkers or agents associated with the disease. For example, aberrant levels of one or more biomarkers or agents in the sample (e.g., levels above a control sample, or above an accepted reference point for the biomarker) indicate the presence of a disease or disorder. In embodiments, the presence of a disease state is identified when greater than 1%, greater than 5%, greater than 25%, greater than 50%, greater than 75%, or greater than 99% of the biomarkers or agents associated with the disease are detected in the sample. In embodiments, the presence of a disease state is identified when about 1%, about 5%, about 25%, about 50%, about 75%, about 99%, or about 100% of the biomarkers or agents associated with the disease are detected in the sample.

In an aspect is provided a method of diagnosing a subject with a disease. In embodiments, the method includes i) providing a collection device to a subject using an unmanned autonomous vehicle; ii) obtaining a sample from the subject and depositing the sample in the collection device; iii) delivering the collection device to an examination center; iv) identifying whether a disease is present in the sample by examining the sample, and v) diagnosing a subject with a disease when the presence of a disease is identified in the sample. In embodiments, the method includes i) providing an unmanned autonomous vehicle; ii) obtaining a sample from the subject and depositing the sample in a collection device; iii) delivering the collection device to an examination center; iv) identifying whether a disease is present in the sample by examining the sample, and v) diagnosing a subject with a disease when the presence of a disease is identified in the sample. In embodiments, the subject uses a collection device (e.g., a collection device the subject purchased from a store, pharmacy, or medical office). In embodiments, the unmanned autonomous vehicle may be requested by the subject. In embodiments, the unmanned autonomous vehicle is provided by a third party (e.g., a physician, or other health care professional) requesting delivery. In embodiments, the method includes treating a subject with a disease.

In some embodiments, the disease is an infectious disease, an autoimmune disease, hereditary disease, or cancer. In embodiments, the disease is an acute disease, a chronic disease (e.g., a malady that exists for greater than 6 months), an idiopathic disease, or a syndrome (e.g., Down syndrome). In embodiments, the disease is a relapsed disease (e.g., a malady that is detectable after a period of time of not being detectable).

In embodiments, the infectious disease is a disease or disorder associated with an infection from a pathogenic organism. In embodiments, the infectious disease is Acinetobacter infections, Actinomycosis, African sleeping sickness (African trypanosomiasis), AIDS (acquired immunodeficiency syndrome), Amoebiasis, Anaplasmosis, Angiostrongyliasis, Anisakiasis, Anthrax, Arcanobacterium haemolyticum infection, Argentine hemorrhagic fever, Ascariasis, Aspergillosis, Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus, Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, Severe acute respiratory syndrome (SARS), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxic shock syndrome (TSS), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, or Zygomycosis.

In embodiments, the disease is an autoimmune disease. In embodiments, the autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, or atopic dermatitis. In embodiments, the autoimmune disease is Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

In embodiments the disease is a hereditary disease. In embodiments, the hereditary disease is cystic fibrosis, alpha-thalassemia, beta-thalassemia, sickle cell anemia (sickle cell disease), Marfan syndrome, fragile X syndrome, Huntington's disease, or hemochromatosis.

In embodiments the disease is a cancer. As used herein, the term “cancer” refers to all types of cancer, neoplasm or malignant tumors found in mammals (e.g., humans), including leukemia, carcinomas and sarcomas. Exemplary cancers that may be treated with a compound or method provided herein include brain cancer, glioma, glioblastoma, neuroblastoma, prostate cancer, colorectal cancer, pancreatic cancer, cervical cancer, gastric cancer, ovarian cancer, lung cancer, and cancer of the head. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof. In embodiments, the cancer is a predefined stage of a breast cancer, a predefined stage of a lung cancer, a predefined stage of a prostate cancer, a predefined stage of a colorectal cancer, a predefined stage of a renal cancer, a predefined stage of a uterine cancer, a predefined stage of a pancreatic cancer, a predefined stage of a cancer of the esophagus, a predefined stage of a lymphoma, a predefined stage of a head/neck cancer, a predefined stage of a ovarian cancer, a predefined stage of a hepatobiliary cancer, a predefined stage of a melanoma, a predefined stage of a cervical cancer, a predefined stage of a multiple myeloma, a predefined stage of a leukemia, a predefined stage of a thyroid cancer, a predefined stage of a bladder cancer, or a predefined stage of a gastric cancer. In some embodiments, the cancer is a predefined subtype of a cancer. In certain instances, the cancer is early stage cancer. In other instances, the cancer is late stage cancer.

In embodiments, the subject is suspected of having a genetic variation or a disease or condition associated with a genetic variation (e.g., an oncogene). In embodiments, the oncogene includes one or more mutations in one or more of the genes TP53, PIK3CA, PTEN, APC, VHL, KRAS, MLL3, MLL2, ARID1A, PBRM1, NAV3, EGFR, NF1, PIK3R1, CDKN2A, GATA3, RB1, NOTCH1, FBXW7, CTNNB1, DNMT3A, MAP3K1, FLT3, MALAT1, TSHZ3, KEAP1, CDH1, ARHGAP35, CTCF, NFE2L2, SETBP1, BAP1, NPM1, RUNX1, NRAS, IDH1, TBX3, MAP2K4, RPL22, STK11, CRIPAK, CEBPA, KDM6A, EPHA3, AKT1, STAG2, BRAF, AR, AJUBA, EPPK1, TSHZ2, PIK3CG, SOX9, ATM, CDKN1B, WT1, HGF, KDM5C, PRX, ERBB4, MTOR, TLR4, U2AF1, ARID5B, TET2, ATRX, MLL4, ELF3, BRCA1, LRRK2, POLQ, FOXA1, IDH2, CHEK2, KIT, HIST1H1C, SETD2, PDGFRA, EP300, FGFR2, CCND1, EPHB6, SMAD4, FOXA2, USP9X, BRCA2, NFE2L3, FGFR3, ASXL1, TGFBR2, SOX17, CDKN1A, B4GALT3, SF3B1, TAF1, PPP2R1A, CBFB, ATR, SIN3A, VEZF1, HIST1H2BD, EIF4A2, CDK12, PHF6, SMC1A, PTPN11, ACVR1B, MAPK8IP1, H3F3C, NSD1, TBL1XR1, EGR3, ACVR2A, MECOM, LIFR, SMC3, NCOR1, RPLS, SMAD2, SPOP, AXIN2, MIR142, RAD21, ERCC2, CDKN2C, EZH2, or PCBP1.

In an aspect is provided a method of detecting a pathogenic organism. In embodiments, the method includes detecting a pathogenic organism in a subject. In embodiments, the method includes i) providing a collection device to a subject using an unmanned autonomous vehicle; ii) obtaining a sample from the subject and depositing the sample in the collection device; iii) delivering the collection device to an examination center; iv) identifying whether a pathogenic organism is present in the sample by examining the sample, and v) detecting a pathogenic organism in a subject when the presence of a pathogenic organism is identified in the sample. In embodiments, the method includes i) providing an unmanned autonomous vehicle; ii) obtaining a sample from the subject and depositing the sample in the collection device; iii) delivering the collection device to an examination center; iv) identifying whether a pathogenic organism is present in the sample by examining the sample, and v) detecting a pathogenic organism in a subject when the presence of a pathogenic organism is identified in the sample. In embodiments, the subject uses a collection device (e.g., a collection device the subject purchased from a store, pharmacy, or medical office). In embodiments, the unmanned autonomous vehicle may be requested by the subject. In embodiments, the unmanned autonomous vehicle is provided by a third party (e.g., a physician, or other health care professional) requesting delivery.

In embodiments, the pathogenic organism. In order to cause disease, pathogenic organisms must be able to enter the host body, adhere to specific host cells, invade and colonize host tissues, and inflict damage on those tissues. In embodiments, the pathogenic organism is bacterium, virus, fungus, protozoa, prion, or helminth (i.e., a parasitic worm).

In embodiments, the pathogenic organism is a gram-negative bacterium (e.g., Salmonella typhi or Yersiniapestis). In embodiments, the pathogenic organism is a gram-positive bacterium (e.g., Staphylococcus aureus, or Clostridium tetani). In embodiments, the pathogenic organism is bacteria that causes a disease. Bacteria include those that cause diseases such as diphtheria (e.g., Corynebacterium diphtheria), pertussis (e.g., Bordetella pertussis), anthrax (e.g., Bacillus anthracia), typhoid, plague, shigellosis (e.g., Shigella dysenteriae), botulism (e.g., Clostridium botulinum), tetanus (e.g., Clostridium tetani), tuberculosis (e.g., Mycobacterium tuberculosis), bacterial pneumonias (e.g., Haemophilus influenzae), cholera (e.g., Vibrio cholerae), salmonellosis (e.g., Salmonella typhi), peptic ulcers (e.g., Helicobacter pylori), Legionnaire's Disease (e.g. Legionella spp.), and Lyme disease (e.g. Borrelia burgdorferi). Other pathogenic bacteria include Escherichia coli, Clostridium perfringens, Clostridium difficile, Pseudomonas aeruginosa, Staphylococcus aureus, and Streptococcus pyogenes. Further examples of bacteria include Staphylococcus epidermidis, Staphylococcus sp., Streptococcus pneumoniae, Streptococcus agalactiae, Enterococcus sp., Bacillus cereus, Bifidobacterium bifidum, Lactobacillus sp., Listeria monocytogenes, Nocardia sp., Rhodococcus equi, Erysipelothrix rhusiopathiae, Propionibacterium acnes, Actinomyces sp., Mobiluncus sp., Peptostreptococcus sp., Neisseria gonorrhoeae, Neisseria meningitides, Moraxella catarrhalis, Veillonella sp., Actinobacillus actinomycetemcomitans, Acinetobacter baumannii, Brucella sp., Campylobacter sp., Capnocytophaga sp., Cardiobacterium hominis, Eikenella corrodens, Francisella tularensis, Haemophilus ducreyi, Helicobacter pylori, Kingella kingae, Legionella pneumophila, Pasteurella multocida, Klebsiella granulomatis, Enterobacteriaceae, Citrobacter sp., Enterobacter sp., Klebsiella pneumoniae, Proteus sp., Salmonella enteriditis, Shigella sp., Serratia marcescens, Yersinia enterocolitica, Yersinia pestis, Aeromonas sp., Plesiomonas shigelloides, Vibrio cholerae, Vibrio parahaemolyticus, Vibrio vulnificus, Acinetobacter sp., Flavobacterium sp., Burkholderia cepacia, Burkholderia pseudomallei, Xanthomonas maltophilia, Stenotrophomonas maltophila, Bacteroides Bacteroides sp., Prevotella sp., Fusobacterium sp., and Spirillum minus.

In embodiments, the pathogenic organism is a virus. Viruses include, but are not limited to, measles, mumps, rubella, poliomyelitis, hepatitis (e.g. hepatitis A, B, C, delta, and E viruses), influenza, adenovirus, rabies, coronavirus, yellow fever, Epstein-Barr virus, herpesviruses, papillomavirus, Ebola virus, influenza virus, Japanese encephalitis, dengue virus, hantavirus, Sendai virus, respiratory syncytial virus, othromyxoviruses, vesicular stomatitis virus, visna virus, cytomegalovirus, and human immunodeficiency virus (HIV).

In embodiments, the pathogenic organism is a fungus. Fungi include, but are not limited to, Acremoniuin spp., Aspergillus spp., Epidermophytoni spp., Exophiala jeanselmei, Exserohilunm spp., Fonsecaea compacta, Fonsecaea pedrosoi, Fusarium oxsporum, Basidiobolus spp., Bipolaris spp., Blastomyces derinatidis, Candida spp., Cladophialophora carrionii, Coccoidiodes immitis, Conidiobolus spp., Cryptococcus spp., Curvularia spp., Fusarium solani, Geotrichum candidum, Histoplasma capsulatum var. capsulatum, Histoplasma capsulatum var. duboisii, Hortaea werneckii, Lacazia loboi, Lasiodiplodia theobromas, Leptosphaeria senegalenisis, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomycete fungi, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Absidia coryinbifera, Rhizomucor pusillus, and Rhizopus arrhizus.

In embodiments, the pathogenic organism is a parasite. Parasites include, but are not limited to, protozoa, nematodes, cestodes, trematodes, and other parasites, such as those responsible for diseases, including, but not limited to, malaria (e.g. Plasmodium falciparum), hookworm, tapeworms, helminths, whipworms, ringworms, roundworms, pinworms, ascarids, filarids, onchocerciasis (e.g., Onchocerca volvulus), schistosomiasis (e.g. Schistosoma spp.), toxoplasmosis (e.g. Toxoplasma spp.), trypanosomiasis (e.g. Trypanosoma spp.), leishmaniasis (Leishmania spp.), giardiasis (e.g. Giardia lamblia), amoebiasis (e.g. Entamoeba histolytica), filariasis (e.g. Brugia malayi), and trichinosis (e.g. Trichinella spiralis).

In an aspect is provided a method of detecting a disease in a subject, the method including: i) requesting transportation of an unmanned autonomous vehicle to a destination; ii) obtaining a sample from a subject and depositing the sample in a collection device; iii) providing the collection device to the unmanned autonomous vehicle; iv) delivering the collection device to an examination center; v) identifying whether a disease is present in the sample by examining the sample, and vi) detecting a disease in a subject when the presence of a disease is identified in the sample. In embodiments, the subject uses a collection device (e.g., a collection device the subject purchased from a store, pharmacy, or medical office). In embodiments, the subject provides the collection device (e.g., a collection device the subject purchased from a store, pharmacy, or medical office). In embodiments, the unmanned autonomous vehicle may be requested by the subject. In embodiments, the unmanned autonomous vehicle is provided by a third party (e.g., a physician, or other health care professional) requesting delivery. In embodiments, the unmanned autonomous vehicle is provided at the request of a third party (e.g., a physician, or other health care professional) requesting delivery.

In embodiments of the methods described herein, providing the collection device to a subject is responsive to a request from the subject, a physician, or other health care professional. In some embodiments, prior to providing a collection device, the method further includes a subject, a physician, other third party (e.g., customs or border agent, TSA agent, employee or contractor for the Department of Defense, affiliated with a public health agency, or acting on the orders of public health officials) or health care professional requesting delivery of a collection through a graphical user interface. In some embodiments, the graphical user interface is a website or an application for use on a mobile computing device. In some embodiments, the mobile computer device is a cellular telephone, a wearable device, a smartphone, a smartwatch, a tablet computer, a personal data assistant (PDA), or a laptop computer.

In some embodiments, prior to providing a collection device, the method further includes a subject, a physician, or other health care professional requesting delivery of a collection device using a computing device. In some embodiments, the computing device is a mobile computing device (e.g., a phone). In embodiments, one or more examination methods are requested at the time the delivery of the collection device is requested.

In some embodiments, the method further includes authenticating the identity of the subject. In some embodiments, the identity of the subject is authenticated by proving a user identity, which includes at least one of a user account, a user password, user certificate information, and user biological feature information. In some embodiments, user certificate information may include a certificate number, a name, the date of birth, a passport number, a social security number, an issuing authority, and a validity period. In embodiments, user certificate information includes the subject's name, date of birth, hospital number, test request form number, accession number, or unique random number. The authentication may occur at any time. In one example, the subject's identity may be authentication prior to collecting the sample. The verification may take place through communications with the medical care provider. For example, authentication may occur within 10 minutes or less, 5 minutes or less, 3 minutes or less, 1 minute or less, 45 seconds or less, 30 seconds or less, 20 seconds or less, 15 seconds or less, 10 seconds or less, 5 seconds or less, 3 seconds or less, 1 second or less, 0.5 seconds or less, or 0.1 seconds or less. The authentication may be automated without requiring any human intervention.

In embodiments, authentication includes analyzing information provided by the subject. For example, the authentication may include scanning an identification card or identification card (e.g., a government issued identification card or insurance card). The authentication may include taking a picture of the subject and/or the subject's face. In embodiments, authentication may include taking a picture of the subject's face or body (e.g., arm, hand, leg, torso, or foot). The authentication may employ a video camera and a microphone that may capture additional visual and audio information. The authentication may include comparing the subject's movements or voice. In embodiments, authentication includes entering personal information related to the subject, such as the subject's name, insurance policy number, answers to key questions, or any other identifying information into a computing device. In embodiments, the authentication includes collecting one or more biometric read-out of the subject (e.g., fingerprint, handprint, footprint, retinal scan, temperature readout, weight, height, or audio information). The biometric information may be collected by the computing device, collection device, or unmanned autonomous vehicle.

In an aspect is provided a method of evaluating a biological sample collected from a subject, the method including: i) requesting delivery of a collection device; ii) providing the collection device to the subject using an unmanned autonomous vehicle; iii) obtaining a biological sample from the subject and depositing the biological sample in the collection device; iv) delivering the collection device to an examination center; v) evaluating the biological sample by examining the biological sample (e.g., examining at the examination center). In embodiments, the method includes evaluating a biological sample collected from a subject at different time points (e.g., every week, every month, or other fixed interval). In embodiments, the method includes evaluating a biological sample collected from a subject daily. In embodiments, the method includes evaluating a biological sample collected from a subject weekly. In embodiments, the method includes evaluating a biological sample collected from a subject monthly. In embodiments, the method includes evaluating a biological sample collected from annually. In embodiments, the method includes repeating steps i) to v).

In an aspect is provided a method of evaluating a biological sample collected from a subject, the method including: i) requesting transportation of an unmanned autonomous vehicle to a destination (e.g., requesting an unmanned autonomous vehicle be provided to a subject); ii) obtaining a sample from a subject and depositing the sample in a collection device; iii) providing the collection device to the unmanned autonomous vehicle; iv) delivering the collection device to an examination center; v) evaluating the biological sample by examining the biological sample.

In some embodiments, the method further includes identifying whether a disease is present in the biological sample by examining the sample. In embodiments, the method further includes detecting a disease in a subject when the presence of a disease is identified in the biological sample.

In some embodiments, the request for delivery includes a request through a graphical user interface. In some embodiments, the request for delivery includes a request using a computing device. In embodiments, the results of examining the sample are communicated to the same computing device. In embodiments, examining includes one or more analyses performed at an examination center.

In some embodiments, examining includes detecting a nucleic acid or biomarker in the sample. In embodiments, the nucleic acid or biomarker is associated with a cancer. In embodiments, the nucleic acid or biomarker associated with a cancer is a nucleic acid or biomarker associated with a gene selected from the group consisting of ACTR3B, ALK, ANLN, AURKA, BAG1, BcI2, BCL2, BCR-Abl, BIRC5, BLVRA, BRAF, c-KIT Cathepsin L2, CCNB1, CCNE1, CD20 antigen, CD30, CD68, CDC20, CDC6, CDH3, CENPF, CEP55, CXXC5, Cyclin Bl, EGFR, ER, ERBB2, ESR1, EXO1, FGFR4, FIP1L-PDGFRalpha, FOXA1, FOXC1, GPR160, GRB7, GSTM1, HOXB13, IL17BR, Ki-67, KIF2C, KRAS, KRT14, KRT17, KRT5, MAPT, MDM2, MELK, MIA, MKI67, MLPH, M1V11³11, MYBL2, MYC, NAT 1, NDC80, NUF2, ORC6L, PDGFR, PGR, PHGDH, PML/RAR alpha, PR, PTTG1, RRM2, SCUBE2, SFRP1, SLC39A6, STK15, Stromelysin 3 (MMP11), Survivin, TMEM45B, TPMT, TYMS, UBE2C, UBE2T, UGT1A1, ABCA10, ABCA9, ADAM33, ADAMTSS, ANGPT1, ANKRD29, ARHGAP20, ARMCX5GPRASP2, ASB1, CA4, CACHD1, CAPN11, CAV1, CAV2, CAV3, CBX7, CCNE2, CD300LG, CDC14B, CDC42SE1, CENPF, CEP68, CFL2, CHL1, CLIP4, CNTNAP3, COL10A1, COL11A1, CRIM1, CXCL3, DAB2IP, DMD, DPYSL2, DST, EEPD1, ENTPD7, ERCC6L, EZH1, F10, FAM126A, FBXO31, FGF1, FIGF, FMO2, FXYD1, GIPC2, GLYAT, GPR17, GPRASP1, GPRASP2, HAGL, HAND2-AS1, HLF, HMMR, HOXA2, HOXA4, HOXAS, IGSF10, INHBA, IL11RA, ITM2A, JADE1, JUN, KIAA0101, KIF4A, KLHL29, LCAT, LGI4, LIFR, LIMS2, LRIG3, LRRC2, LRRC3B, MAMDC2, MATN2, MICU3, MIR99AHG, MME, MMP11, NECAB1, NEK2, NKAPL, NPHP3, NR3C1, NR3C2, NUF2, PAMR1, PAFAH1B3, PAQR4, PARK2, PEAR1, PGMS, PKMYT1, PLEKHM3, PLSCR4, POU6F1, PPAP2B, PPP1R12B, PRCD, PRX, PYCR1, RAPGEF3, RBMS2, SCN4B, SDPR, SLC35A2, SH3BGRL2, SPRY2, STATSB, SYN2, TK1, TMEM220, TMEM255A, TMOD1, TPM3, TPX2, TSHZ2, TSLP, TSTA3, TTC28, WISP1, USHBP1, USP44, IBSP, CST1, ZWINT, and combinations thereof.

In some embodiments, examining includes performing an immunoassay, nucleic acid assay, receptor-based assay, nucleic acid sequencing, cytometry, a colorimetric assay, an enzymatic assay, a spectroscopic assay, electrophoresis, an agglutination assay, chromatography, coagulation assay, an electrochemical assay, histological method, a nephelometric assay, a turbidimetric assay, a radioisotope assay, viscometric assay, a protein synthesis assay, culture assay, osmolality assay, or a combination of any of the foregoing. In some embodiments, examining includes performing an immunoassay selected from the group radioimmunoassay, counting immunoassay, enzyme immunoassay, enzyme-linked immunosorbent assays, fluoroimmnoassay, electrochemiluminescenceimmunoassay, or chemiluminescenceimmunoassay. In embodiments, examining includes performing polymerase chain reaction (PCR), quantitative PCR (QPCR), next generation sequencing, RNAseq, digital PCR (dPCR), digital droplet PCR (ddPCR), isothermal amplification, endoribonucleotide strand displacement assay (ERiN SDA), microarray, and combinations thereof. In embodiments, examining includes performing nucleic acid sequencing (e.g., an SBS sequencing method). In embodiments, examining includes performing RNAseq. In embodiments, examining includes performing quantitative (QPCR).

In embodiments, examining includes determining whether a pathogenic organism is present in a sample. Various methods may be used to determine the presence of a pathogenic organism. For example, a sample may be used in an immunoassay to assay for an antigen which is present in a pathogen of interest. Alternatively, an immunoassay may involve using an antigen to detect the presence of an antibody in the sample generated against a pathogen. Immunoassays include, for example, ELISAs, immunoblotting, and antibody-based cell-staining for microscopy. In embodiments, the sample is assayed for a bacterial marker, such as the 16S ribosomal (small subunit) RNA (16S rRNA) or its gene (16S rDNA), 23 S rRNA, rpoB, gyrB, dnaK, amoA, or mip genes. In embodiments, the sample is assayed for a fungal marker, such as the internal transcribed spacer (ITS) region of the ribosomal cistron (as described in Schoch, et. al. PNAS Apr. 17, 2012 109 (16) 6241-6246, for example).

In embodiments, examining includes observation, measurement and analysis of a sample by cytometry. Cytometry may include preparing and analyzing images of cells in a biological sample (e.g., two-dimensional images), where the cells are labeled (e.g., with fluorescent, chemiluminescent, enzymatic, or other labels), placed on a substrate, and imaged by a camera. The camera may be attached to or used in conjunction with a microscope. Cells may be identified in the two-dimensional images by their attached labels (e.g., from light emitted by the labels).

In embodiments, examining includes performing IR spectroscopy. IR spectroscopy may further be used to measure substances in blood samples, such as alcohol levels, fatty acid content, cholesterol levels, glucose, and hemoglobin concentration. IR spectroscopy can also distinguish between synovial fluid from healthy and arthritic patients.

In embodiments, examining includes a colorimetric assay. Colorimetry refers to measuring the transmissive color absorption of a subject, preferably by backlighting the subject with white light with the result sensed by an imaging sensor. Examples include some assays that use oxidases or peroxidases combined with a dye that becomes colored in the presence of hydrogen peroxide. Colorimetric assays may be used to detect analytes that include but are not limited to alcohols, cholesterols, lactate, uric acid, glycerol, triglycerides, glutamate, glucose, choline, NADH and use enzymes such as horseradish peroxidase, lactoperoxidase, microperoxidase, alcohol oxidase, cholesterol oxidase, NADH oxidase.

In some embodiments, the examining includes performing a Clinical Laboratory Improvement Amendments (CLIA)-waived test.

In some embodiments, examining the sample includes culturing at least a portion of the sample with a microorganism growth medium and a metabolic indicator, wherein the metabolic indicator is capable of being metabolized to produce a metabolic product; and detecting the metabolic product. In some embodiments, the metabolic indicator includes resazurin, 5-cyano-2,3-ditolyl tetrazolium chloride (CTC), carboxyfluorescein diacetate succinimidyl ester (CFDA-SE), or luciferin.

In some embodiments, examining the sample includes performing a nucleic acid assay, wherein the nucleic acid assay includes contacting at least one primer complementary to a portion of a nucleic acid within the sample. In some embodiments, examining the sample includes detecting the presence of antibodies to the pathogenic organism. In some embodiments, examining the sample includes sequencing a nucleic acid sequence, or complement thereof, in the sample.

In some embodiments, delivering the collection device to an examination center is performed using an unmanned autonomous vehicle. In some embodiments, delivering the collection device to an examination center is performed using the same unmanned autonomous vehicle that provided the collection device.

In some embodiments, the examining center is a laboratory. In some embodiments, the examining center is a Clinical Laboratory Improvement Amendments (CLIA)-compliant laboratory or a CLIA-certified laboratory. In embodiments, the examination center is a hospital, a clinical laboratory improvement amendments (CLIA) lab, an operating room, or a central facility that serves an operating room, a non-CLIA lab, an emergency room, a specialized care unit, a hospital ward, a mobile care site, an outpatient clinical suite such as, e.g., an outpatient surgical suite, a veterinary care center, outpatient facility, permanent or temporary structure, including a field unit, in a vehicle, for example, an automobile, airplane, helicopter, train, ship, boat, submarine, or ambulance, in a home or office, a food or beverage processing facility, a slaughterhouse, or a farm. In embodiments, examining is performed by a qualified individual (e.g., a person possessing legitimate skill, experience, education, or other requirements of an employment position at an examination center).

In some embodiments, the sample includes blood, serum, saliva, urine, tears, gastric and/or digestive fluid, stool, mucus, sputum, sweat, earwax, oil, glandular secretion, semen, or vaginal fluid. In embodiments, the sample includes food, hair, nails, skin, or clothing. In embodiments, the sample includes whole blood, plasma, serum, cerebrospinal fluid, ascites, sweat, tears, saliva, urine, buccal sample, semen, vaginal fluid, cavity rinse, food sample, or organ rinse. In embodiments, the sample includes vomitus, feces, serum, or urine.

In embodiments, the sample may contain or is suspected of containing one or more pathogens (e.g., a pathogenic organism). Examples of samples may include but are not limited to, blood, serum, saliva, urine, gastric and digestive fluid, tears, stool, semen, vaginal fluid, interstitial fluids derived from tumorous tissue, ocular fluids, sweat, mucus, earwax, oil, glandular secretions, breath, spinal fluid, hair, fingernails, skin cells, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk or other excretions. A sample may be provided from, for example, a human, animal, plant, microorganism, the environment, food, or from other sources. In embodiments, a sample may contain material obtained from a swab from a subject (e.g. a nasal swab).

In some embodiments, the sample includes a nasopharyngeal swab sample, a nasal aspirate sample, or a nasal wash sample. In some embodiments, the sample includes bronchial aspirate, endotracheal aspirate, or tracheal aspirate. In some embodiments, the sample includes dermal interstitial fluid. In some embodiments, the sample is a nasopharyngeal swab sample, a nasal aspirate sample, or a nasal wash sample. In some embodiments, the sample is bronchial aspirate, endotracheal aspirate, or tracheal aspirate. In some embodiments, the sample is dermal interstitial fluid.

In embodiments, the sample includes bacteria, viral particles, proteins, prions, remnants or portions thereof. In embodiments, the sample includes an analyte, biomarker, or nucleic acid. In embodiments, the sample includes drugs, prodrugs, pharmaceutical agents, drug metabolites, biomarkers such as expressed proteins and cell markers, antibodies, antigens, proteins, hormones, polypeptides, glycoproteins, polysaccharides, lipids, viruses, cholesterol, polysaccharides, nucleic acids, genes, nucleic acids, and combinations thereof.

In embodiments, the sample is a fluid. In embodiments, a volume of sample may be provided from the subject. Examples of volumes may include, but are not limited to, about 10 mL or less, 5 mL or less, 3 mL or less, 1 microliter (μL) or less, 500 μL, or less, 300 μL, or less, 250 μL, or less, 200 μL, or less, 170 μL, or less, 150 μL, or less, 125 μL, or less, 100 μL, or less, 75 μL, or less, 50 μL, or less, 25 μL, or less, 20 μL, or less, 15 μL, or less, 10 μL, or less, 5 μL, or less, 3 μL, or less, 1 μL, or less, 500 nL or less, 250 nL or less, 100 nL or less, 50 nL or less, 20 nL or less, 10 nL or less, 5 nL or less, 1 nL or less, 500 pL or less, 100 pL or less, 50 pL or less, or 1 pL or less. In embodiments, the volume of a sample is about 1 to 10 mL.

In embodiments, the sample is a biological sample. As used herein, the terms “biological sample,” and “patient sample,” are interchangeably used and refer to any sample taken from a subject, which can reflect a biological state associated with the subject. In some embodiments such samples contain cell-free nucleic acids such as cell-free DNA (cfDNA). In some embodiments, such samples include nucleic acids other than or in addition to cell-free nucleic acids. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject. The biological sample can include any tissue or material derived from a living or dead subject.

In embodiments, library preparative steps may be performed during transit or at the examining center, including cell lysing, RNA or DNA extraction and purification, primer ligation, amplification, etc., such that the sample may be sequenced according to known techniques in the art. For example, the biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. In embodiments, the collection device is capable of disrupting the tissue or cell structure. Disrupting the cell may include lysing the cell, sonicating the cell, homogenizing the cell, shaking the cell, vortexing a solution containing the cell, and combinations thereof. Lysing the sample may include contacting the sample with a lysing agent, wherein the lysing agent is in a solution or the lysing agent is a solution. Lysing agents may include one or more detergents, such as CHAPS, CHAPSO, sodium dodecyl sulfate

(SDS), ethyl trimethyl ammonium bromide, Triton-X 100, Triton X-114, NP-40, Brij-35, Brij-58, Tween-20, Tween 80, octyl glucoside, and octyl thioglucoside. Lysing agents may include chaotropic agents, such as guanidinium isothiocyanate, urea, butanol, ethanol, guanidinium chloride, lithium perchlorate, lithium acetate, magnesium chloride, phenol, propanol, sodium dodecyl sulfate, and thiourea.

In embodiments, the method includes performing an amplification reaction (e.g., an isothermal amplification, a reverse transcription reaction, and a combination thereof). In embodiments, the isothermal amplification and reverse transcription reaction may occur in the collection unit, wherein the reverse transcription reaction transcribes RNA in the sample to produce a cDNA, wherein the cDNA is the target nucleic acid. The isothermal amplification may be include Loop-mediated Isothermal Amplification (LAMP), Helicase-Dependent Amplification (HDA), Recombinase Polymerase Assay (RPA), Transcription-Mediated Amplification (TMA), Nucleic Acid Sequence-Based Amplification (NASBA), Signal mediated amplification of RNA Technology (SMART), Strand Displacement Amplification (SDA), Rolling Circle Amplification (RCA), Isothermal Multiple Displacement Amplification (IMDA), Single Primer Isothermal Amplification (SPIA), Recombinase Polymerase Assay (RPA), or Self-sustained Sequence Replication (3 SR).

In embodiments, the collection device includes components necessary to obtain a sample (e.g., reagents (e.g., ethanol), distilled water, swabs (e.g., sterilizing swabs, collection swabs, Moore swabs), cotton, spoons, scoops, tongue depressor, forceps, tongs, spatula, pipettes, sponges, containers, and/or plastic bags. In some embodiments, the collection device includes a microfluidic paper-based analytical device (μPAD), cotton swab, transdermal patch, or device configured to collect and store a bodily fluid. In some embodiments, the collection device includes a nasal swab, nasopharyngeal swab, oropharyngeal swab, throat swab, buccal swab, oral fluid swab, stool swab, tonsil swab, vaginal swab, cervical swab, blood swab, or wound swab. In some embodiments, the collection device includes a swab, at least one vessel, a buffer, or preservative. In embodiments, the collection device includes a squeegee or cell scraper. In embodiments, the collection device includes a sample collection tube, an Eppendorf, a container, or any device that is suitable for storing biological materials. In embodiments, the collection device is a container. In embodiments, the collection device is a nasopharyngeal swab. In embodiments, the collection device is a container configured to receive a swab.

In embodiments, the collection device includes reagents useful to perform one or more of the chemical reactions and/or other preparative steps. For example, for a luminogenic ELISA assay the reagents within the collection device may include a sample diluent, a detector conjugate (e.g., enzyme-labeled antibodies), a surface labeled with antibodies binders, a wash solution, and an enzyme substrate. Enzyme-linked ImmunoSorbent assays (“ELISA”) are assays using antibodies to bind a target analyte in a solution or on a substrate. For example, collection devices may include substrates having adherent antibodies or target antigens, which may be subjected to examination and/or detection at an examination center.

In embodiments, the collection device includes a surface. The surface may have a coating that promotes adhesion of the sample to the surface. In embodiments, the coating includes poly-1-lysine, poly-d-lysine, poly-ornithine, a collagen, a laminin, a fibronectin, a mucopolysacharride, heparin sulfate, hyaluronidate, chondroitin sulfate, or a hydrogel. In embodiments, the coating may be used to selectively or non-selectively bind cells. The coating may selectively bind one or more specific cell types, e.g., ductal, epithelial, or glandular cells. For example, the coating may be selected to bind to certain cell types such as T-cell or B-cell, but not to adipocytes. In embodiments, surface includes a coating which selectively binds cells that express a specific marker or set of markers on a cell surface.

In embodiments, the collection device is used to extract, isolate, and hold or carry the sample. For example, nasal swabs, throat swabs, stool samples, hair, finger nail, ear wax, breath, and other solid, semi-solid, or gaseous samples may be processed in an extraction buffer, e.g., for a fixed or variable amount of time, prior to their analysis. In embodiments, the collection device includes reagents inside a receptacle to release the sample into the reagents. Reagents include without limitation wash buffers, enzyme substrates, dilution buffers, conjugates, enzyme-labeled conjugates, DNA amplifiers, sample diluents, wash solutions, detergents, polymers, chelating agents, albumin-binding reagents, enzyme inhibitors, enzymes, anticoagulants, red-cell agglutinating agents, or antibodies. In embodiments, the reagents include a weak base (e.g., tris-hydroxymethyl amino methane (Tris), ethanolamine, triethanolamine, glycine, and alkaline salts of organic acids, such as trisodium citrate), a chelating agent (e.g., EDTA), an anionic detergent (e.g., sodium dodecyl sulfate (SDS) and sodium lauryl sarcosinate (SLS)), and/or a preservative. In embodiments, the collection device includes a slide, a plate, a tube, a chip, a nitrocellulose membrane, or a paper. In embodiments, the collection device includes a slide, swab, tube, vial, container, chip, paper, or plate. In embodiments, the collection device is made of any suitable material, for example glass, plastic (e.g., polystyrene, polypropylene, or other plastic), film, a nanofiber matrix, a cellulose matrix (e.g., filter paper), or other solid substance. In embodiments, the collection device is a swab.

In some embodiments, the sample may be a blood sample. A first container may receive whole blood and a second container may receive blood plasma. Anticoagulants (e.g., EDTA or citrate salts) may be provided along the fluid path and/or in the containers. In embodiments, the same includes cell free nucleic acids. As used herein, the term “cell free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell free nucleic acids originate from one or more healthy cells and/or from one or more cancer cells Cell free nucleic acids are used interchangeably as circulating nucleic acids. Examples of the cell-free nucleic acids include but are not limited to RNA, mitochondrial DNA, or genomic DNA. As used herein, the terms “cell free nucleic acid,” “cell free DNA,” and “cfDNA” are used interchangeably. As used herein, the term “circulating tumor DNA” or “ctDNA” refers to nucleic acid fragments that originate from tumor cells or other types of cancer cells, which may be released into a fluid from an individual's body (e.g., bloodstream) as result of biological processes such as apoptosis or necrosis of dying cells or actively released by viable tumor cells.

In some embodiments, the collection device includes a container with integrated thermal control unit or material that provides active or passive cooling. In embodiments, the thermal control material is an embedded phase change material (PCM) material that maintains the temperature at a prior, or desired temperature. For example, the phase change material can oppose changes in temperature around the critical temperature where the material would undergo a phase change. If the PCM is embedded, the collection device and the passive cooling element may be one and the same. In embodiments, the collection device includes active cooling system. In embodiments, unmanned autonomous vehicle is capable of controlling the temperature of the collection device. For example, the unmanned autonomous vehicle maintains the temperature between −20° C. and −70° C. In embodiments, the unmanned autonomous vehicle maintains the temperature between 4° C. and 20° C. In embodiments, this temperature range is between about 1 to 10° C., optionally 2 to 8° C., or between 2 to 6° C.

In some embodiments, the collection device includes a container outside the collection device to provide further physical protection and/or thermal control capability. For example, the container can be in the form of a corrugated plastic box, inflatable air pillows, corrugated carboard, plastic, or fiber.

In some embodiments, any number of containers may be provided within the sample collection device. The sample containers may be capable of receiving sample received from a subject. Each sample container may have a unique identifier. The unique identifier may be associated with any information relating to the sample, subject, device, or component of the device. In some embodiments, each identifier for each container may be unique. In other embodiments, the identifier on the container need not be unique, but may be unique for the device, for the subject, or for the type of sample.

A sample collection device may receive a sample from a subject. The subject may directly contact the sample collection device or provide the sample to the device. The sample may travel through the device to one or more containers within the device. In some embodiments, the sample may be treated prior to reaching the containers. One or more coating or substance may be provided within a sample collection unit and/or channel that may convey the sample to the containers. Alternatively, no treatment is provided to the sample prior to reaching the container. In some embodiments, the sample may or may not be treated within the container. In some embodiments, a plurality of different types of treatments may be provided to a sample before or when the sample reaches the container. The treatments may be provided in a preselected order. For example, a first treatment desired first, and may be provided upstream of a second treatment. In some embodiments, the sample is not treated at any point.

In embodiments, the collection device further includes inlet and outlet ports, syringes, openings which may be connect to valves, tubes, channels and/or pumps. In some cases, the collection device is capable of being connected to controllers with programmable valves to move fluids and/or the sample through the device.

In some embodiments, the collection device includes an identifiable tag. In some embodiments, the identifiable tag is a label, an RFID, or an EEPROM. The identifiers may be read by any suitable technique. By way of example and not limitation, in some embodiments, the identifiers are read using an optical detector, such as an image capture device or barcode scanner. In one example, an image capture device may capture an image of a QR code. Information relating to the container may be tracked. For example, when a container arrives at a location, the identifier may be scanned, and record of the arrival of the container may be kept. The progress and/or location of the container may be updated actively and/or passively. In some embodiments, the identifier may need to be scanned intentionally in order to determine the location of the container. In other examples, the identifier may actively emit a signal that may be picked up by signal readers. For example, as an identifier travels through a building, signal readers may track the location of the identifier.

In some embodiments, reading the identifier may permit a user to access additional information associated with the identifier. For example, the user may capture an image of the identifier using a device. The device or another device may display information about the sample, subject, device, component of the device, or any other information described elsewhere herein. Information about tests to be conducted and/or test results may be included. The user may perform subsequent tests or actions with the sample based on information associated with the identifier. For example, the user may direct the container to the appropriate location for a test. In some embodiments, the container may be directed to an appropriate location and/or have appropriate sample processing (e.g., sample prep, assay, detection, analysis) performed on the contents of the container in an automated fashion without requiring human intervention.

Information relating to sample processing may be collected and associated with the identifier. For example, if a container has an identifier and sample processing has been performed on the contents of the container, one or more signals produced in response to the sample processing may be stored and/or associated with the identifier. Such updates may be made in an automated fashion without requiring human intervention. Alternatively, a user may initiate the storing of information or may manually enter information. Thus, medical records relating to a subject may be aggregated in an automated fashion. The identifiers may be useful for indexing and/or accessing information related to the subject.

In embodiments, the collection device includes a self-sterilization modality. For example, following sample collection from the subject, and during transport to an examination center, the unmanned autonomous vehicle (UAV) may suffer a travel disruption that may compromise the mission and may expose the collection device to the environment. While the sample collection device may contain buffers and/or solutions that neutralize any pathogens or potentially biohazardous material in the sample, depending on the sample type, there may still be biologically active substances in the collection device. Additionally, the collection device may contain genetic information in the sample that could be used by a third party to identify the subject, or which may be tampered with and invalidate subsequent testing. In embodiments, the travel disruption may activate the self-sterilization modality. The self-sterilization modality destroys or inactivates any biological material in the collection device that may contain identifying information about the subject.

A self-monitoring system regarding the UAV's health (e.g., mechanical/electrical systems status) and/or a sensor for detecting any deviation from a pre-approved flight plan may determine whether the self-sterilization modality is activated. In embodiments, the travel disruption includes loss of power, mechanical failure, electrical failure, or a sudden drop in speed. In embodiments, the travel disruption includes a sudden change in temperature, detection of elevated humidity, detection of an abnormally high level of smoke (e.g., carbon monoxide), or detection of a physical shock to the UAV. In embodiments, the travel disruption does not include physical damage to the UAV. In embodiments, the travel disruption includes repeated attempts to access the collection device and/or cargo area of the UAV. In embodiments, the travel disruption includes unauthorized access to the collection device and/or cargo area of the UAV. In embodiments, the travel disruption includes removal of any UAV component (e.g., battery, engine, wing, wheels, collection device, etc.) before arriving at a destination. In embodiments, the self-sterilization modality is powered through an independent power source.

In embodiments, the self-sterilization modality includes a DNA-damaging agent. For example, in embodiments, the DNA-damaging agent includes UV light or a mild bleach (e.g., 0.05% sodium hypochlorite solution), which upon detection of a travel disruption, is activated and/or released into the collection device and contacted with the sample. Contact between the DNA-damaging agent and the sample thus neutralizes any biological material, as well as destroys any genetic material to preserve patient privacy in the event that the UAV is intercepted by an unauthorized party. In embodiments, the self-sterilization modality includes heat, an acid, a base, UV light, radiation, a DNAse, a RNAse, or a combination thereof. In embodiments, the self-sterilization modality includes self-generated plasma fields (see, e.g., U.S. Pat. Pub. 2018/0008737, which is incorporated herein by reference in its entirety). In embodiments, the self-sterilization modality includes a plurality of UV light emitting sources (see, e.g., U.S. Pat. Pub. 2019/0365938, which in incorporated herein by reference in its entirety). In embodiments, the collection device includes a sensor to indicate that the sterilization process has been performed and completed successfully. Completion of the sterilization process may be assessed by using an indicator system to measure a selected property of the self-sterilization modality, for example, exposure to UV light or exposure to a base. In embodiments, the sterilization indicator includes a growth medium for detecting bacterial growth (see, e.g., U.S. Pat. Pub. 2010/0081165, which is incorporated herein by reference in its entirety).

In some embodiments, the method further includes providing a report to the subject, a healthy agency, a physician, or other health care professional, wherein the report includes the results from examining the sample. In some embodiments, the report is provided in less than 24 hours from depositing the sample in the collection device. In some embodiments, the method further includes transmitting data to a computing device or web server, wherein the data includes data associated with examining the sample. In some embodiments, the data is transmitted in less than 24 hours from depositing the sample in the collection device. In embodiments, the report includes identifying information about the individual, healthy agency, a physician, or other health care professional, and optionally the type and conditions (e.g., temperature and time) for the analyses/examination methods performed at the examination center.

The systems and methods described herein enable rapid analysis of samples and provide results rapidly. For instance, the systems and methods described herein may produce the result(s) in less than about 12, less than about 11, less than about 10, less than about 9, less than about 8, less than about 7, less than about 6, less than about 5, less than about 4, less than about 3, less than about 2, or less than about 1 hour from sample collection. In embodiments, the systems and methods described herein may produce the result(s) in less than about 12, less than about 11, less than about 10, less than about 9, less than about 8, less than about 7, less than about 6, less than about 5, less than about 4, less than about 3, less than about 2, or less than about 1 hour from receipt of the sample at the examination center.

In embodiments, the systems and methods described herein may produce the result(s) in less than about 48, less than about 47, less than about 46, less than about 45, less than about 44, less than about 43, less than about 42, less than about 41, less than about 40, less than about 39, less than about 38, less than about 37, less than about 36, less than about 35, less than about 34, less than about 33, less than about 32, less than about 31, less than about 30, less than about 29, less than about 28, less than about 27, less than about 26, less than about 25, less than about 24, less than about 23, less than about 22, less than about 21, less than about 20, less than about 19, less than about 18, less than about 17, less than about 16, less than about 15, less than about 14, less than about 13, less than about 12, less than about 11, less than about 10, less than about 9, less than about 8, less than about 7, less than about 6, less than about 5, less than about 4, less than about 3, less than about 2 hours from receipt of the sample at the examination center. In embodiments, the systems and methods described herein may produce the result(s) in about 5 to about 12 hours, about 1 to about 6 hours, about 0.5 to about 2 hours, about 20 to about 60 minutes, about 10 to about 30 minutes from receipt of the sample at the examination center.

In embodiments, the report includes a complete, partial, and/or summary of the examination results. The report includes results from the examination center. The results include the outcome of the one or more assays or experiments described herein, such as for example results from an immunoassay, nucleic acid assay, receptor-based assay, nucleic acid sequencing, cytometry, a colorimetric assay, an enzymatic assay, a spectroscopic assay, electrophoresis, an agglutination assay, chromatography, coagulation assay, an electrochemical assay, histological method, a nephelometric assay, a turbidimetric assay, a radioisotope assay, viscometric assay, a protein synthesis assay, culture assay, or osmolality assay.

In embodiments, the method further includes communicating therapy or treatment options in response to a diagnosis or detection event to a subject, a physician, or other health care professional. In embodiments, the therapy or treatment, includes a drug, a diet, a radiation treatment, a chemotherapeutic agent, a biological therapeutic, an injection, a physical therapy, or exercise. The methods may further include communicating with a subject, a physician, or other health care professional for prescription of chemotherapy, radiotherapy, endocrine therapy, or combinations thereof.

In embodiments, the method includes communicating with a subject, a physician, or other health care professional for prescription an antibiotic. Antibiotics are agents used to kill, inhibit, or slow the growth of bacteria or other microorganisms and include, but are not limited to, aminoglycosides (such as amikacin, gentamicin, kanamycin, neomycin, netilmicin, tobramycin, paromomycin, streptomycin, and spectinomycin), ansamycins (such as geldanamycin, herbimycin, and rifaximin), carbacephem (such as loracarbef) carbapenems (such as ertapenem, doripenem, imipenem/cilastatin, and meropenem), cephalosporins (such as cefadroxil, cefaxolin, cefalotin (cefalothin), cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil and ceftobiprole), glycopeptides (such as teicoplanin, vancomycin, telavancin, dalbavancin, and oritavancin), lincosamides (such as clindamycin and lincomycin), lipopetides (such as daptomycin), macrolides (such as azithromycin, clarithromycin, dirithromycin, erythromycin, roxithromycin, troleandomycin, telithromycin, and spiramycin), monobactams (such as aztreonam), nitrofurans (such as furazolidone, and nitrofurantoin), oxazolidinones (such as linezolid, posizolid, radezolid, and torezolid), penicillins (such as amoxicillin, ampicillin, azlocillin, carbenicillin, cloxacillin, dicloxacillin, flucloxacillin, mexlocillin, methicillin, nafcillin, oxacillin, penicillin G, penicillin V, piperacillin, temocillin, and ticarcillin), polypeptides (such as bacitracin, colistin, and polymyxin B), quinolones/fluoroquinolones (such as ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, and temafloxacin), sulfonamides (such as mafenide, sulfacetamide, sulfadiazine, silver sulfadiazine, sulfadimethoxine, sulfamethizole, sulfamethoxazole, sulfanilamide, sulfasalazine, sulfisoxazole, trimethoprim-sulfamethoxazole, and sulfonamidochrysoidine), tetracylines (such as demeclocycline, doxycycline, minocycline, oxytetracycline, and tetracycline), antimycobacteria (such as clofazimine, dapsone, capreomycin, cycloserine, ethambutol, ethionamide, isoniazid, pyrazinamide, rifampicin, rifabutin, rifapentine, streptomycin), arsphenamine, chloramphenicol, fosfomycin, fusidic acid, metronidazole, mupirocin, platensimycin, quinupristin/dalfopristin, thiamphenicol, tigecycline, tinidazole, trimethoprim, and teixobactin.

In embodiments, the method includes communicating with a subject, a physician, or other health care professional for prescription an antiviral. Antivirals are agents used to kill, inhibit, or slow the growth of viruses and include, but are not limited to, anti-(HIV) agents (such as abacavir, didanosine, emtricitabine, lamivudine, stavudine, tenofovir disoproxil fumarate, zidovudine, delavirdine, efavirenz, etravirine, nevirapine, rilpivirine, atazanavir, darunavir, fosamprenavir, indinavir, nelfinavir, ritonavir, saquinavir, tipranavir, enfuvirtide, maraviroc, dolutegravir, elvitegravir, raltegravir, cobicistat, and combinations thereof), anti-influenza virus agents (such as zanamivir, oseltamivir phosphate, peramivir, amantadine, and rimantadine), anti-herpes virus agents (such as acyclovir, valacyclovir, penciclovir, idoxuridine, vidarabine, trifluridine, foscarnet and famciclovir), anti-hepatitis virus agents (such as adefovir, lamivudine, telbivudine, tenofovir, famciclovir, entecavir, ribavirin, telaprevir, simeprevir, sofosbuvir, ledipasvir, ombitasvir, paritaprevir, ritonavir, dasabuvir, and boceprevir), anti-cytomegalovirus (CMV) agents (such as ganciclovir, cidofovir, valganciclovir, foscarnet, maribavir, and leflunomide), anti-respiratory syncytial virus (RSV) agents (such as ribavirin and palivizumab), and anti-varicella-zoster virus (VSV) agents (such as acyclovir, valacyclovir, penciclovir, famciclovir, brivudin, foscarnet, and vidarabine).

In embodiments, the method includes communicating with a subject, a physician, or other health care professional for prescription an antifungal. Antifungals are agents used to kill, inhibit, or slow the growth of fungi and include, but are not limited to, polyene antifungals (such as amphotericin B, candicin, filipin, hamycin, natamycin, nystatin, and rimocidin), azole antifungals (such as abafungin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, and terconazole, voriconazole), and echinocandins (such as anidulafungin, caspofungin, and micofungin).

In embodiments, the method includes communicating with a subject, a physician, or other health care professional for prescription an antiparasitic. Antiparasitics are agents used to kill, inhibit, or slow the growth of parasites and include, but are not limited to, antinematodes (such as mebendazole, pyrantel pamoate, thiabendazole, diethylcarbamazine, and ivermectin), anticestodes (such as niclosamide, praziquantel, and albendazole), antitrematodes (such as praziquantel), antiamoebics (such as rifampin and amphotericin B), and antiprotozoals (such as melarsoprol, eflornithine, metronidazole, tinidazole, and miltefosine).

In an aspect is provided a method of providing a collection device to a subject, the method including: i) receiving from a subject a request a request for delivery of one or more collection devices; ii) providing one or more collection devices to the subject using an unmanned autonomous vehicle, wherein the one or more collection devices include a biological collection device.

In some embodiments, the unmanned autonomous vehicle is an unmanned aerial vehicle.

III. Kits, Systems, and Computing Devices

In an aspect is provided a kit including a collection device. Generally, the kit includes one or more containers providing composition and one or more additional reagents (e.g., a buffer suitable for sample collection). In some embodiments, the kit is provided to a subject using an unmanned autonomous vehicle. In some embodiments, the kit includes instructions for sample collection. In embodiments, the kit includes instructions and information on fasting, diet, and medication restrictions. In embodiments, the kit includes reagents (e.g., ethanol), sterilizing swabs, a marking pen, cotton, distilled water, spoons, scoops, tongue depressor, forceps, tongs, spatula, pipettes, Moore swabs (i.e., gauze strips), sponges, containers, and/or plastic bags. In embodiments, the kit includes an ice pack.

In embodiments, the collection device is a collection device described herein. For example, a collection device may include a suitable vessel, container, or material, such as a microfluidic paper-based analytical device (μPAD), cotton swab, transdermal patch, or device configured to collect and store a fluid or biological sample. In some embodiments, the collection device includes a nasal swab, nasopharyngeal swab, oropharyngeal swab, throat swab, buccal swab, oral fluid swab, stool swab, tonsil swab, vaginal swab, cervical swab, blood swab, or wound swab.

In embodiments, the kits include a control (alternatively referred to as a standard). The control may be utilized to detect and/or confirm the presence of a control biomarker, a control nucleic acid, or a control analyte. The control nucleic acid may include a nucleic acid selected from genomic DNA, mitochondrial DNA, chloroplast DNA, microbial DNA, cDNA, messenger RNA, ribosomal RNA, micro RNA, an amplicon thereof, and a combination thereof. The control nucleic acid may encode pre-determined internal reference genes against which the target nucleic acid(s) are compared to obtain a normalization ratio. A plurality of control nucleic acids may comprise a control nucleic acid signature. The control nucleic acid signature may indicate a cell type. The method may be a control for homogenizing and/or lysing the cell within a sample.

In embodiments, the kit includes a plurality of primers for PCR amplifying and/or for sequencing nucleic acids isolated from the sample. The kit may provide at least 1, 2, 3, 4, 5, 6, 7, 8, 9, 10, 20, 50, 100, 200, 500, 1000, or more primers. The kit may provide between about 1-3, 1-10, 5-20, 1-1000, 10-500, 20-200, or 50-100 primers. In embodiments, the primers include 5, 10, 15, 20, 25, 30, 40, 50, 100, 150, 200 or more nucleotides.

In embodiments, the kit includes instructions to perform a test using reagents from other vendors. For example, the kit may instruct users to use a Qiagen purification kit to isolate mRNA from the cellular samples collected using the provided sample collection device. In embodiments, the kit includes spin column technology (e.g., RNeasy Plus Micro Kit) or magnetic bead-based technology (e.g. ARCTURUS® PicoPure® RNA Isolation Kit, Dynabeads® mRNA DIRECT1υ Micro Kit) that may isolate mRNA, total RNA, or total nucleic acids from the sample.

In an aspect is provided a computing device configured to enable the evaluation of a biological sample collected from a subject, including: a processor; memory in electronic communication with the processor; and instructions stored in the memory, the instructions being executable by the processor to: a. request for delivery of a collection device; b. communicate with a server to provide the collection device using an unmanned autonomous vehicle; c. communicate with a server to deliver the collection device containing a biological sample to an examination center; d. receive data associated with the evaluation of the biological sample from the examination center.

In embodiments, the computing device is a personal computer system, server computer system, hand-held or laptop device, multiprocessor system, microprocessor-based system, set top box, programmable consumer electronic, network PC, minicomputer system, mainframe computer system, smartphone, or distributed cloud computing environments that include any of the above systems or devices. The computing device can include one or more processors or processing units, a memory architecture that may include RAM and non-volatile memory. The memory architecture may further include removable/non-removable, volatile/non-volatile computer system storage media. Further, the memory architecture may include one or more readers for reading from and writing to a non-removable, non-volatile magnetic media, such as a hard drive, a magnetic disk drive for reading from and writing to a removable, non-volatile magnetic disk, and/or an optical disk drive for reading from or writing to a removable, non-volatile optical disk such as a CD-ROM or DVD-ROM.

In embodiments, the computing device includes memory in electronic communication with the processor. The memory architecture may include at least one program module implemented as executable instructions that are configured to carry out one or more steps of a method set forth herein. For example, executable instructions may include an operating system, one or more application programs, other program modules, and program data. Generally, program modules may include routines, programs, objects, components, logic, and data structures that perform particular tasks. A computing device can optionally communicate with one or more external devices such as a keyboard, a pointing device (e.g., a mouse), a display, such as a graphical user interface (GUI), or other device that facilitates interaction of a use with the unmanned autonomous vehicle. Similarly, the computing device can communicate with other devices (e.g., via network card, modem, etc.). Such communication can occur via I/0 interfaces. In embodiments, the computing system may communicate with one or more networks such as a local area network (LAN), a general wide area network (WAN), and/or a public network (e.g., the Internet) via a suitable network adapter.

In an aspect is provided a system, including: at least one data processor; and at least one memory storing instructions, which when executed by the at least one data processor, result in operations including: receiving, from a client device, a request for a collection device; responding to the request by at least identifying a first unmanned autonomous vehicle for delivering the collection device, the first unmanned autonomous vehicle being identified based at least on a first location associated with the request and/or a second location of the first unmanned autonomous vehicle; sending, to the first unmanned autonomous vehicle, a first command to deliver the collection device to the first location; and sending, to the first unmanned autonomous vehicle or a second unmanned autonomous vehicle, a second command to deliver the collection device including a sample from the first location to an examination center.

In some embodiments, the system includes an operation to verify the identity of a client, the operation includes receiving, from a client device, authenticating information and comparing the authenticating information to pre-collected authenticating information, wherein a match between the authenticating information and said pre-collected authenticating information verifies the identity of the client.

In embodiments, the system may verify the identity of the subject. The verification may be performed by the collection device. The verification may be performed by the unmanned autonomous vehicle. The verification may be performed by the computing device. The verification may occur at any time. In one example, the subject's identity may be verified prior to collecting the sample. The verification may take place through communications with the medical care provider, laboratory, payer, laboratory benefits manager, or any other entity. Verification may occur by accessing one or more data storage units. The data storage units may include an electronic medical records database. Verification may occur rapidly and/or in real-time. For example, verification may occur within 10 minutes or less, 5 minutes or less, 3 minutes or less, 1 minute or less, 45 seconds or less, 30 seconds or less, 20 seconds or less, 15 seconds or less, 10 seconds or less, 5 seconds or less, 3 seconds or less, 1 second or less, 0.5 seconds or less, or 0.1 seconds or less. The verification may be automated without requiring any human intervention.

In embodiments, verification includes information provided by the subject. For example, the verification may include scanning an identification card or identification card (e.g., a government issued identification card or insurance card). The verification may include taking a picture of the subject and/or the subject's face. For example, the verification may include taking a two-dimensional or three-dimensional image of the subject. Cameras may be used which may provide a two-dimensional digital image of the subject and/or that may be capable of formulating a three-dimensional or four-dimensional image of the subject. A four-dimensional image of the subject may incorporate changes over time. In embodiments, verification may include taking a picture of the subject's face or body (e.g., arm, hand, leg, torso, or foot). The verification may employ a video camera and a microphone that may capture additional visual and audio information. The verification may include comparing the subject's movements or voice.

In embodiments, verification includes entering personal information related to the subject, such as the subject's name, insurance policy number, answers to key questions, or any other identifying information into a computing device. In embodiments, the verification includes collecting one or more biometric read-out of the subject (e.g., fingerprint, handprint, footprint, retinal scan, temperature readout, weight, height, or audio information). The biometric information may be collected by the computing device, collection device, or unmanned autonomous vehicle.

In embodiments, verification (i.e., authenticating) includes the use of a public key infrastructure (PKI) to encrypt the verification information provided by the subject. PKI refers to set of roles, policies, and procedures to create, manage, distribute, use, store, and revoke digital certificates for public-key encryption. As designed, PKI includes (1) a global certificate authority (“CA”) that issues and signs digital certificates, (2) a global directory authority (“DA”) that stores digital certificates and lists of revoked digital certificates, indexes the digital certificates, and delivers digital certificates to requestors, and (3) a global registration authority (“RA”) that verifies the identity of an entity (e.g., a person or organization) who requests their digital certificate to be stored at the DA. A PKI may be implemented using a blockchain for improved scalability, for example, for storing all public keys (see, e.g., U.S. Pat. Pub. 2018/0006826, which is incorporated herein by reference in its entirety). For example, the verification may include public key cryptography, wherein the subject possesses both a private key and a public key. In public key cryptography, the private key decrypts a message encrypted with the public key, and vice versa. The public key, for example, is recognized as the subject's own personal key and is freely available on servers, and is associated with any contact information disclosed by the subject. With a PKI, encrypted messages including verification information may be sent after encrypting with the public key, and subsequently decrypted with the private key. In another non-limiting example, a public key embedded in a digital certificate may be digitally signed by an accepted authority (e.g., DigiCert, Thawte, etc.) to affirm its authenticity. In embodiments, the public key infrastructure includes a digital copy of a manifest including a description of the collection device. In embodiments, the digital copy of the manifest is digitally signed. In embodiments, the digital copy of the manifest is timestamped and digitally signed. In embodiments, the digital copy of the manifest is timestamped, digitally signed, and stored in a trusted third party database. A private key may also be combined with a unique device ID or identifier for proving who has received or sent particular protected data, as described in U.S. Pat. Pub. 2009/0150674, which is incorporated herein by reference in its entirety).

In embodiments, and for the purposes of maintaining confidentiality, a digitally signed hash code or hash function may be transmitted to a trusted third party. A hash function is a function that converts data to an encrypted form. Methods of using hash functions in digital signature services for authentication of user information are known, and detailed, for example, in U.S. Pat. Nos. 7,310,617, 9,819,494, and 10,091,004, each of which is incorporated herein by reference in its entirety. In embodiments, the hash function is a one-way function in order to prevent reversal of the output to recover the input. For example, the one-way hash function allows one to share evidence that one possesses some information (e.g., verification information), but doesn't disclose what that information is. In embodiments, the verification information provided by the subject are computed and stored. The hash function as computed by the computer would then be compared with what's stored in a database and authenticated. In embodiments, verification includes the use of a public key infrastructure wherein a digital copy of a manifest including a description of the collection device is timestamped, digitally signed, and a digitally signed hash code is transmitted to a trusted third party to be timestamped and digitally signed, and stored in the trusted third party database. In embodiments, the verification information is authenticated by signing with a private key. In embodiments, the verification information is encrypted with the use of a hash code (e.g., hash function, such as a one-way hash function). In embodiments, the verification information is encrypted with a hash function, timestamped, digitally signed, and transmitted to a trusted third party. The trusted third party may then decrypt the authenticating information. In embodiments, a secondary hash function is computed from the primary hash function.

In some embodiments, the first location is determined based on a location of the first client device. In some embodiments, the first location is determined based on a location provided as part of the request. In some embodiments, the first unmanned autonomous vehicle is identified from a plurality of unmanned autonomous vehicles as being configured for the collection device. In some embodiments, the first unmanned autonomous vehicle is identified from a plurality of unmanned autonomous vehicles as being in a location that is the closest to the first location. In some embodiments, the first unmanned autonomous vehicle is identified from a plurality of unmanned autonomous vehicles as having the capacity to receive the collection device. In some embodiments, the second command is in response to loading of the collection device. In some embodiments, the system further includes an operation for tracking the location of the first unmanned autonomous vehicle, the second unmanned autonomous vehicle, and/or the collection device.

In an aspect is provided an unmanned autonomous vehicle, including: at least one data processor; and at least one memory storing instructions, which when executed by the at least one data processor, result in operations including: sending a first message including a current location of the unmanned autonomous vehicle; receiving a command to deliver a collection device to a location or to deliver the collection device from the location to an examination center; and in response to detecting a deposit and/or a removal of the collection device at the location, sending a second message including an updated location of the unmanned autonomous vehicle and/or an updated capacity of the unmanned autonomous vehicle. In embodiments, sending the first message includes the current capacity of the unmanned autonomous vehicle. In embodiments, sending the first message includes sending the message to a central server. In embodiments, sending the first message includes sending the message to a second autonomous drone.

In some embodiments, the memory further stores a device ID corresponding to the identity of the unmanned autonomous vehicle and activity log.

In an aspect is provided a system, including: an unmanned autonomous vehicles; and a central server communicatively coupled with the unmanned autonomous vehicle, the central server including at least one data processor and at least one memory, the at least one memory may store instructions, which when executed by the at least one data processor, result in operations including: receiving, from a client device, a request for a collection device; receiving, from the unmanned autonomous vehicle, a message including a current location of the unmanned autonomous vehicle; responding to the request by at least identifying the unmanned autonomous vehicle for delivering the collection device, the unmanned autonomous vehicle being identified based at least on a location associated with the request and the current location of the unmanned autonomous vehicle; and in response to identifying the unmanned autonomous vehicle for delivering the collection device, sending, to the unmanned autonomous vehicle, a command to deliver the collection device to the location associated with the request.

In some embodiments, the location associated with the request is provided using a graphical user interface. In embodiments, the graphical user interface includes a map of the location.

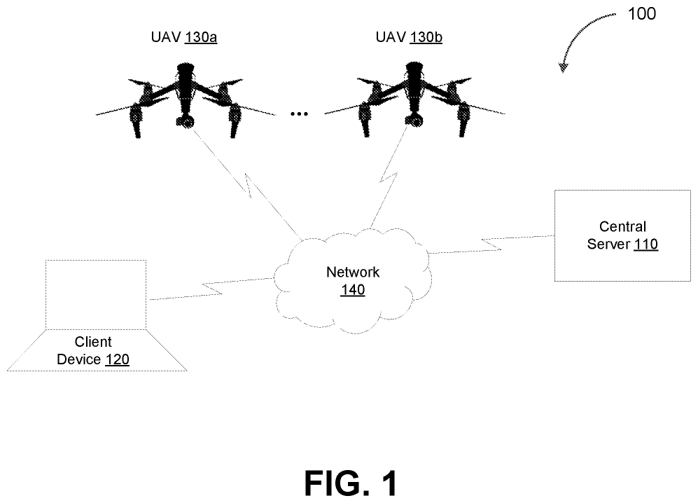

FIG. 1 depicts a system diagram illustrating an example of an unmanned autonomous vehicle biological specimen evaluation system 100, in accordance with some example embodiments. Referring to FIG. 1, the unmanned autonomous vehicle biological specimen evaluation system 100 may include a central server 110, a client device 120, and one or more unmanned autonomous vehicles 130. As shown in FIG. 1, the central server 110, the client device 120, and the one or more unmanned autonomous vehicles 130 may be communicatively coupled via a network 140. The client device 120 may be a processor based device including, for example, a smartphone, a tablet computer, a wearable apparatus, a virtual assistant, an Internet-of-Things (IoT) appliance, and/or the like. The one or more unmanned autonomous vehicles 130 may be any type of vehicle of sensing and navigating its environment with minimal to no operator guidance. The network 140 may be any wired and/or wireless network including, for example, a public land mobile network (PLMN), a wide area network (WAN), a local area network (LAN), a virtual local area network (VLAN), the Internet, and/or the like.

In some example embodiments, the central server 110 may respond to receiving, from the client device 120, a request for a collection device by at least identifying and dispatching a first unmanned autonomous vehicle 130a for delivering the collection device to a first location associated with the request. Moreover, the central server 110 may respond to the request for the collection device by at least identifying and dispatching the first unmanned autonomous vehicle 130a or a second unmanned autonomous vehicle 130b to retrieve the collection device including a biological sample from the first location associated with the request and deliver the collection device including the biological sample to an examination center.

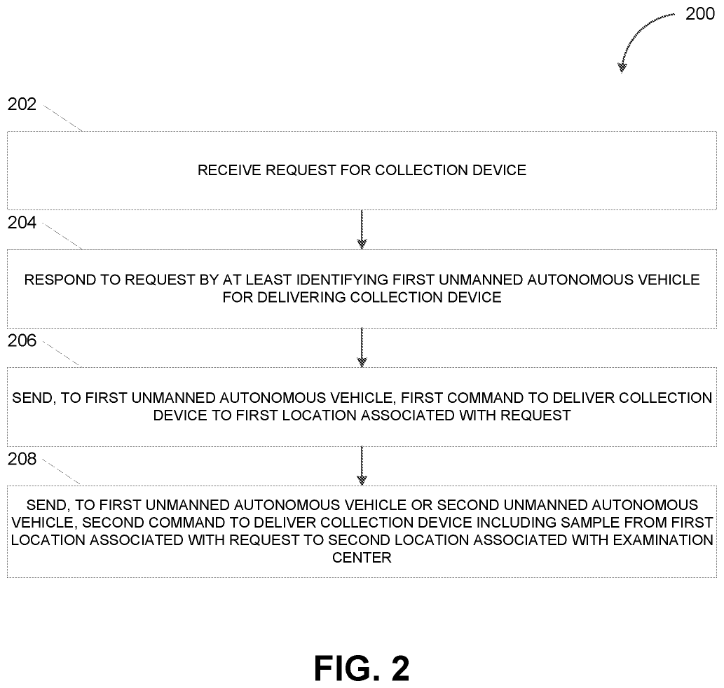

FIG. 2 depicts a flowchart illustrating an example of a process 200 for evaluating a biological sample, in accordance with some example embodiments. Referring to FIGS. 1-2, the process 200 may be performed by the central server 110. As shown in FIG. 2, at 202, the central server 110 may receive a request for a collection device. For example, the central server 110 may receive, from the client device 120, a request for a collection device. The request may be associated with a first location at which to deliver (and retrieve) the collection device. At 204, the central server 110 may respond to the request by at least identifying a first unmanned autonomous vehicles for delivering the collection device. In some example embodiments, for example, the central server 110 may identify the first unmanned autonomous vehicle 130a (instead of the second unmanned autonomous vehicle 130b ) based at least on the first location associated with the request and/or a second location of the first unmanned autonomous vehicle 130a. At 206, the central server 110 may send, to the first unmanned autonomous vehicle, a first command to deliver the collection device to a first location associated with the request. At 208, the central server 110 may send, to the first unmanned autonomous vehicle or a second unmanned autonomous vehicle, a second command to deliver the collection device including a sample from the first location associated with request to a second location associated with an examination center. For instance, the central server 110 may dispatch the first unmanned autonomous vehicle 130a to deliver the collection device to the first location associated with the request. Moreover, the central server 110 may dispatch either the first unmanned autonomous vehicle 130a or the second unmanned autonomous vehicle 130b to retrieve the collection device including a biological sample from the first location associated with the request and deliver that collection device to an examination center.

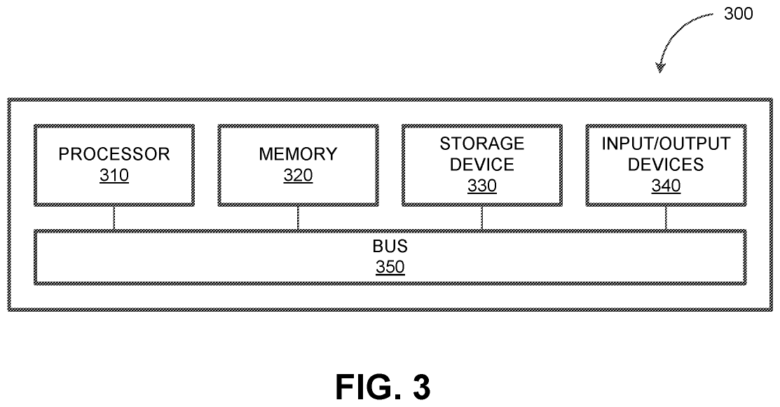

FIG. 3 depicts a block diagram illustrating an example of a computing system 300, in accordance with some example embodiments. Referring to FIGS. 1-3, the computing system 300 can be used to implement the central server 110 and/or any components therein.

As shown in FIG. 3, the computing system 300 can include a processor 310, a memory 320, a storage device 330, and an input/output device 340. The processor 310, the memory 320, the storage device 330, and the input/output device 340 can be interconnected via a system bus 350. The processor 310 is capable of processing instructions for execution within the computing system 300. Such executed instructions can implement one or more components of, for example, the central server 110 and/or the like. In some example embodiments, the processor 310 can be a single-threaded processor. Alternately, the processor 310 can be a multi-threaded processor. The processor 310 is capable of processing instructions stored in the memory 320 and/or on the storage device 330 to display graphical information for a user interface provided via the input/output device 340.

The memory 320 is a computer readable medium such as volatile or non-volatile that stores information within the computing system 300. The memory 320 can store data structures representing configuration object databases, for example. The storage device 330 is capable of providing persistent storage for the computing system 300. The storage device 330 can be a floppy disk device, a hard disk device, an optical disk device, or a tape device, or other suitable persistent storage means. The input/output device 340 provides input/output operations for the computing system 300. In some example embodiments, the input/output device 340 includes a keyboard and/or pointing device. In various implementations, the input/output device 340 includes a display unit for displaying graphical user interfaces.

According to some example embodiments, the input/output device 340 can provide input/output operations for a network device. For example, the input/output device 340 can include Ethernet ports or other networking ports to communicate with one or more wired and/or wireless networks (e.g., a local area network (LAN), a wide area network (WAN), the Internet).

In some example embodiments, the computing system 300 can be used to execute various interactive computer software applications that can be used for organization, analysis and/or storage of data in various formats. Alternatively, the computing system 300 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 340. The user interface can be generated and presented to a user by the computing system 300 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

### EXAMPLES

### Example 1

### Contactless Diagnosis of an Infection

Patients showing symptoms of a disease or condition are often required to go to a medical office to receive a diagnosis and treatment. Infectious organisms are ubiquitous in a medical environment, despite vigorous efforts to maintain antisepsis. The presence of these organisms can result in further infection of other patients and medical personnel. The simple act of traveling to a medical office, such as a physician clinic, urgent care facility, or emergency room when infected with a communicable disease (e.g., influenza, tuberculosis, SARS-CoV-1, or SARS-CoV-2) potentially exposes hundreds if not thousands of individuals to the infection and risks the safety and wellbeing of others. Transmission of an infectious disease occurs when an infectious organism moves from one host to another and causes disease in the new host. There are many factors that contribute to and influence transmission, however it is known that transmission can occur via contact, either direct contact with a fomite, or indirect contact with droplets or aerosols from expiratory releases (e.g., coughing, sneezing, or speaking). Limiting, or reducing, contact with the environment outside your own home is known to reduce the transmission and spread of contagions. Moreover, it is essential to receive results of a test to confirm the presence of a contagion as rapidly as possible, which is a challenge under the current health care system. For example, see the potential risks and expected turnaround times in Table 1 and Table 2.

TABLE 1
Non-limiting comparative example of the analysis pipeline

| | | Methods and Systems |
|---|---|---|
| Analysis step | Traditional Methods | described herein |
| Sample collection: | 1-3 days: scheduling an | <3 hours: user |
| obtaining the | appointment, going to | requests a drone and |
| specimen | office/lab to collect | provides a test kit; drone |
| | the sample, transporting | delivers the sample to an |
| | sample to analysis center | analysis center |
| Sample Prep | 1-10 days: centrifugation, | 1-6 hours: isolation, |
| (e.g., isolation, | enrichment, incubation, | extraction, |
| purification, | cell culture | amplification |
| enrichment) | | |
| Diagnostic: | 1-48 hours | 2-3 hours: rapid |
| sequencing, assays, | | sequencing, |
| qtPCR | | rapid diagnostics |
| Analysis: compiling | 1-24 hours | 1 hour |
| raw results to | | |
| conclude a diagnosis | | |
| Report: reporting to | 1-2 days | 1 hour: report is |
| the individual and/or | | transmitted electronically |
| physician | | following analysis |

TABLE 2
Summary of the Potential Risks.

| | | Methods and Systems |
|---|---|---|
| Risk | Current urgent care | described herein |
| Potential exposure to | Significant risk | No risk |
| other sick people | | |
| Potentially infecting | Significant risk | No risk |
| others | | |
| Travel to facility | Required | Not required |
| Range of available | Limited | Access to full menu of |
| tests | | lab tests |
| Hours of operation | Limited | Unlimited |
| Results reporting | Typically by telephone | Securely communicated |
| | or in person | via a computing device |
| Ability to | Not currently | Yes |
| consult specialist | available | |
| via telemedicine | | |
| Results | Some are immediate; | Rapid turnaround, |
| | others require | for example results |
| | send-out | are provided between |
| | & may take days | 1 hr and 1 day, |
| | | depending on test |
| Cost | High-requires | Low |
| | multiple health | |
| | professionals (e.g., | |
| | nurses and doctors) | |

An individual is interested in learning if he or she has an infectious disease (e.g., influenza or SARS-CoV-2). From their home, they access a website or use an application on their smart device to request a test kit to be delivered to their home. The test kit is delivered via an unmanned aerial vehicle (UAV) colloquially referred to as a drone, for example an FAA certified drone to handle medical tissue, such as those described by Matternet, Amazon, or the UPS Flight Forward™ programs. Alternatively, the individual may have obtained a test kit, via alternative means (e.g., purchasing the kit at a store, pharmacy, or medical office). The individual collects the sample using the test kit (e.g., a nasopharyngeal swab), provides the test kit to a drone, and the drone delivers it to an examination center. Timing is important, especially for RNA viruses. Degradation can occur quickly in samples, so the time between sample collection and examining (e.g., sequencing) should be minimized. Ideally, the sample is placed in a transportation and preservation solution, such as UTM™ or CyMol™ (Copan), to maintain nucleic acid integrity and inactivate pathogen infectivity. Due to flight limitations of drone technology and sensitivity of the sample, the examination center may be located within 100 miles of the individual and referred to as a regional examination center. The drone may deliver the sample to a temporary holding facility where it is further delivered to an examination center, optionally via non-drone means.

Various library preparative steps may be performed during transit or at the examining center, including cell lysing, RNA or DNA extraction and purification, primer ligation, amplification, etc., such that the sample may be sequenced according to known techniques in the art. For example, depending on whether the sample includes RNA or DNA, either an RNA or DNA library may be prepared. Viral RNA libraries are prepared using QlAamp Viral RNA extraction kit (Qiagen) as per the manufacturer's instructions. Briefly, host DNA is removed by DNase treatment followed by inactivation at 80° C. The RNA is transcribed to cDNA using Protoscript II kit (New England Biolabs) with the supplied random primer mix followed by second strand DNA synthesis using a such enzymes as Escherichia coli DNA ligase, DNA polymerase I, and RNase H (New England Biolabs). DNA libraries are prepared similarly as for RNA libraries, omitting a DNase pre-treatment and cDNA synthesis steps. Libraries are constructed with the barcoded, platform specific primers.

Once at the examination center, the sample is analyzed for the presence of a biomarker indicative of an infectious disease. Examining may include one or more assays (e.g., immunoassay, nucleic acid assay, receptor-based assay, nucleic acid sequencing, cytometry, a colorimetric assay, an enzymatic assay, a spectroscopic assay, electrophoresis, an agglutination assay, chromatography, coagulation assay, an electrochemical assay, histological method, a nephelometric assay, a turbidimetric assay, a radioisotope assay, viscometric assay, a protein synthesis assay, culture assay, osmolality assay). Examining may include nucleic acid sequencing. Nucleic acid sequencing may be completed using known techniques such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing comprises a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template.

In embodiments, the genomic sequence is obtained. In embodiments, a partial genomic sequence is obtained (e.g., a gene sequence). Enough of the genomic sequence is acquired to determine the presence and/or identity of a pathogenic organism. Genomic alignments may be performed using known techniques in the art. General methods for performing sequence alignments are known to those skilled in the art. Examples of suitable alignment algorithms, include but are not limited to the Needleman-Wunsch algorithm (see e.g. the EMBOSS Needle aligner available at www.ebi.ac.uk/Tools/psa/emboss_needle/, optionally with default settings), the BLAST algorithm (see e.g., the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/, optionally with default settings). Optimal alignment may be assessed using any suitable parameters of a chosen algorithm, including default parameters. Assembly algorithms known in the art can align and merge overlapping sequence reads generated by methods of several embodiments herein to provide a contiguous sequence of a nucleic acid of interest. A person of ordinary skill in the art will understand which sequence assembly algorithms or sequence assemblers are suitable for a particular purpose taking into account the type and complexity of the nucleic acid of interest to be sequenced (e.g. genomic, PCR product, or plasmid), the number and/or length of deletion products or other overlapping regions generated, the type of sequencing methodology performed, the read lengths generated, whether assembly is de novo assembly of a previously unknown sequence or mapping assembly against a backbone sequence, etc. Furthermore, an appropriate data analysis tool will be selected based on the function desired, such as alignment of sequence reads, base-calling and/or polymorphism detection, de novo assembly, assembly from paired or unpaired reads, and genome browsing and annotation. In several embodiments, overlapping sequence reads can be assembled by sequence assemblers, including but not limited to ABySS, AMOS, Arachne WGA, CAP3, PCAP, Celera WGA Assembler/CABOG, CLC Genomics Workbench, CodonCode Aligner, Euler, Euler-sr, Forge, Geneious, MIRA, miraEST, NextGENe, Newbler, Phrap, TIGR Assembler, Sequencher, SeqMan NGen, SHARCGS, SSAKE, Staden gap4 package, VCAKE, Phusion assembler, Quality Value Guided SRA (QSRA), Velvet (algorithm), and the like. It will be understood that overlapping sequence reads can also be assembled into contigs or the full contiguous sequence of the nucleic acid of interest by available means of sequence alignment, computationally or manually, whether by pairwise alignment or multiple sequence alignment of overlapping sequence reads. Algorithms suited for short-read sequence data may be used in a variety of embodiments, including but not limited to Cross_match, ELAND, Exonerate, MAQ, Mosaik, RMAP, SHRiMP, SOAP, SSAHA2, SXOligoSearch, ALLPATHS, Edena, Euler-SR, SHARCGS, SHRAP, SSAKE, VCAKE, Velvet, PyroBayes, Pb Short, and ssahaSNPAdditional phylogenetic analyses can be conducted using Mauve (Darling, A. C. et al Genome Res 14, 1394-1403 (2004)) or MAFFT (Katoh, K. & Standley, D. M. Mol Biol Evol 30, 772-780 (2013)).

Variant calling may be provided by known methods in the art. Sequence differences between viral genomes mark the evolutionary history and relationships between samples. Single-base substitutions (single nucleotide polymorphisms, or SNPs) are the simplest variants. Given high-quality consensus sequences aligned to a reference, it is relatively easy to manually identify SNPs. However, more complex approaches—such as those implemented in packages like GATK (McKenna A. et al. Genome Res. 2010; 20(9):1297-1303) or Samtools (Li H. et al. Bioinformatics. 2009; 25(16):2078-79) are helpful when samples contain insertions or deletions, or if regions of the genome have poor quality, low coverage, or high diversity

Pathogenicity is not only dependent on qualitative issues such as the presence of specific species, strains, or genes, but also on their relative abundances. Therefore, the method further includes quantifying the relative abundance of a pathogen, or pathogenic material (e.g., a toxin). If a positive result for an infectious organism is detected, it may be recommended to the individual to perform a follow-up test in order to confirm the initial test results. Alternatively, if the library or sequencing reads did not meet certain quality metrics, then the health care provider and/or laboratory analyzing the sample would initiate a second round of sample collection via the UAV platform. Communication and scheduling of the follow-up test kit delivery is performed through the same interface the individual accessed initially.

The results are then securely communicated to the individual, and optionally any medical staff as requested by the individual. The results may be communicated via email, text message, phone call, or other means, physical or electronic. The methods described herein may be a useful adjunct to telemedicine. For example, if the results are communicated with the individual's physician, appropriate diagnosis and treatment options may begin immediately. Depending on the nature of the infectious organism detected (e.g., a contagious BSL-3 or BSL-4 pathogen), additional public health responses may be taken. In particular, the present invention would facilitate the rapid initiation of contact tracing efforts through the individual's local health department. For example, in combination with state-of-the-art contact tracing tools on mobile devices, users with whom the contagious individual has been in contact with could be notified and given the option to receive a test kit by a UAV.

Alternatively, or additionally, the results are securely communicated to the individual's smartphone. The smartphone may then present a certificate, or passport, certifying the individual is not infected. The passport may then expire, prompting the individual to get retested.

Minimizing the turn-around time from test request to results is paramount. Utilizing state of the art sequencers and technology, in embodiments, the methods described herein provide sequencing results in less than 24 hours from receipt of the sample at the examination center. This has considerable potential to enable real-time surveillance of circulating pathogenic strains so as to facilitate rapid and appropriate response to outbreaks.

### Example 2

### Detecting Waterborne Pathogens

Waterborne disease is a global burden which is estimated to cause more than 2.2 million deaths per year and higher cases of illness every day, including diarrhea, gastrointestinal diseases, and systematic illnesses (Ramirez-Castillo FY et al. Pathogens (2015) 4(2): 307-334). In some embodiments, the waterborne pathogens may include bacteria such as Salmonella typhimurium, Vibrio cholerae, and Legionella, viruses such as adenoviruses, parvoviruses, and coronaviruses, or protozoan agents such as Crytosporidium, Naegleria fowleri, and Giardia. Rapid detection of such waterborne pathogens will facilitate public health goals in both rural and developing regions.

An individual lacking a water supply that is monitored and treated by a municipality is interested in learning if he or she has waterborne pathogens present in their water supply. The individual may access a website or use an application on their smart device to request a test kit to be delivered to their home. The test kit is delivered via an unmanned aerial vehicle (UAV) colloquially referred to as a drone, for example an FAA certified drone to handle medical tissue, such as those described by Matternet, Amazon, or the UPS Flight Forward™ programs. Alternatively, the individual may have obtained a test kit, via alternative means (e.g., purchasing the kit at a store, pharmacy, or medical office). The individual collects the water sample using the test kit (e.g., one or more collection tubes) and a drone delivers it to an examination center. The same drone that provided the test kit may wait as the sample is collected. Alternatively, the individual may have acquired a collection kit and uses a website or use an application on their smart device to request a drone be delivered to their home to collect the test kit. Degradation can occur quickly in samples, so the time between sample collection and examination should be minimized. Due to flight limitations of drone technology and sensitivity of the sample, the examination center may be located within 100 miles of the individual and referred to as a regional examination center. The drone may deliver the sample to a temporary holding facility where it is further delivered to an examination center, optionally via non-drone means.

Processing of the water sample(s) may vary depending on the type of pathogen being screened. Sequencing-based methods as described in Example 1 may be applied to total nucleic acid isolates for microbiome analyses. Alternatively, or in addition, samples may be cultured in defined growth mediums to enrich for specific class(es) of microorganism(s). Bacterial cultures, for example, would allow for alternative tests such as antibiotic susceptibility testing. In order to screen for the presence of the faecal-associated pathogen Clostridium difficile, immunoassays for toxin A and B, and glutamate dehydrogenase (GDH) may be performed on cultures from the water sample (Giuliano C et al, P T (2019) 44(4): 192-200).

The results are then securely communicated to the individual, and optionally any medical staff as requested by the individual. The results may be communicated via email, text message, phone call, or other means, physical or electronic. Minimizing the turn-around time is paramount. Utilizing state of the art sequencers and technology, in embodiments, the methods described herein provide sequencing results in less than 24 hours from receipt of the sample at the examination center. This has considerable potential to enable real-time surveillance of circulating waterborne pathogenic strains so as to facilitate rapid and appropriate response to outbreaks.

### Example 3

### Environmental Metagenomics

It is estimated that between 60 and 75 percent of emerging infectious diseases in humans come from other animals (Jones et al. Nature. 2008; 451(7181): 990-993; Taylor et al.; Philos. Trans. R Soc. Lond. B Biol Sci. 2001 Jul 29; 356(1411): 983-989). Referred to as zoonoses, the pathogens responsible for rabies, Lyme disease, anthrax, mad cow disease, SARS, Ebola, West Nile, Zika, Q fever, orf, Rift Valley fever, and Kyasanur Forest disease, for example, pose significant health issues as the increased frequency and speed of travel (e.g., local and international), the increase in human-assisted movement of animals and animal products, and environmental changes facilitate the transmission of infectious agents. Sequencing of viral samples can supplement epidemiological methods by providing nucleotide-level resolution of outbreak-causing pathogens. For example, when the World Health Organization issued a global alert for SARS in 2003, the disease-causing agent was still unknown. Subsequent sequencing of isolates led to the identification of SARS coronavirus (SARS-CoV) as the pathogen responsible (Lipsitch M, Cohen T, Cooper B, Robins J M, Ma S, et al. Science. 2003; 300(5627):1966-70) More recently, metagenomic RNA sequencing of a sample of bronchoalveolar lavage fluid from a patient allowed scientists to identify the novel SARS-CoV-2 virus in late January 2020, (Wu F, Zhao S, Yu B, et al. Nature. 2020 Apr;580(7803):E7] and Nature. 2020;579(7798):265-269). Knowing the identity of the pathogen allows researchers to improve diagnostics and methods of treatment. Thus, effective genomic analysis of viral outbreaks relies on comprehensive sample collection and rapid sequencing.

Providing greater access to sequencing to scientists and field researchers will vastly improve our understanding of the local environment, and in the case of viral outbreaks, enhance the response by enabling mitigating measures to limit transmission. For example, a deadly pathogen of finfish is the viral hemorrhagic septicemia virus (VHSV), a member of the genus Novirhabdovirus, and in 2003 it was found in the Laurentian Great Lakes causing serious losses in a number of important fish species (Faisal M. Viruses. 2012 May; 4(5): 734-760). Scientists responded by developing a quantitative reverse-transcriptase polymerase chain reaction (qRT-PCR) assay (Hope K M, et al. J Aquat. Anim. Health. 2010;22(1):50-61) and gained more insight into the viral diversity by examining the partial gene sequence (Thompson T M, et al. Dis. Aquat. Organ. 2011; 96(1):29-43). Ongoing sequencing efforts discovered a virulent substrain of VHSV, VHSV-IVb which due to novel mutations continues to evolve allowing it to evade fish host recognition and immune responses (Stepien C A et al. PLoS One. 2015;10(8):e0135146).

### Example 4

### Immunochemical Fecal Occult Blood Testing

Blood in stool is an indication of a number of gastrointestinal disorders, including diverticulitis, colitis, polyps, and colorectal cancer. The American Cancer Society and Centers for Disease Control and Prevention recommend a fecal occult blood test annually after age 50 to aid in the early detection of colorectal cancer. Immunochemical fecal occult blood (iFOB) tests for at-home specimen collection are commercially available, such as Quidel's QuickVue® iFOB Test, a one-step lateral flow chromatographic immunoassay containing anti-hHb antibodies conjugated with colloidal gold for the detection of human hemoglobin.

An individual is interested in learning if he or she has the presence of blood in their stool. From their home, they access a website or use an application on their smart device to request an iFOB test kit to be delivered to their home. The test kit is delivered via an unmanned aerial vehicle (UAV) colloquially referred to as a drone, for example an FAA certified drone to handle medical tissue, such as those described by Matternet, Amazon, or the UPS Flight Forward™ programs. The individual collects the specimen using the test kit (e.g., a collection tube and sampler) and a drone delivers it to an examination center. Degradation can occur quickly in samples, so the time between sample collection and examining should be minimized. Due to flight limitations of drone technology and sensitivity of the sample, the examination center may be located within 100 miles of the individual and referred to as a regional examination center. The drone may delivery the sample to a temporary holding facility where it is further delivered to an examination center, optionally via non-drone means.

The results are then securely communicated to the individual, and optionally any medical staff as requested by the individual. The results may be communicated via email, text message, phone call, or other means, physical or electronic. Minimizing the turn-around time is paramount. Utilizing state of the art technology, in embodiments, the methods described herein provide iFOB test results in less than 24 hours from receipt of the sample at the examination center. Positive results can lead to early detection of colorectal cancer, and lead to additional treatment plans for the individual.

### Example 6

### Chronic Disease Monitoring

Long-term treatment with warfarin is recommended for patients with atrial fibrillation at risk of stroke and those with recurrent venous thrombosis or prosthetic heart valves (Tideman P A et al. Aust. Prescr. 2015; 38(2): 44-48). Regular monitoring of the anticoagulant effect is required. Patients on warfarin and their physicians must constantly balance the risks of bleeding and clotting (Jailer A and Bragg L Cleve. Clin. J. Med. 2003; 70(4): 361-71). Evidence suggests that patients who self-monitor using point-of-care testing have better outcomes than other patients (Matchar D B et al. N. Engl. J. Med. 2010; 363:1608-20). A prothrombin time test is commonly used to determine the international normalized ratio (INR) and monitor the effectiveness of warfarin treatment.

An individual initiating or on maintenance with warfarin is interested in monitoring his or her blood clotting function. From their home, they access a website or use an application on their smart device to request an INR test kit, for example, a prothrombin time test kit, to be delivered to their home. The test kit is delivered via an unmanned aerial vehicle (UAV) colloquially referred to as a drone, for example an FAA certified drone to handle medical tissue, such as those described by Matternet, Amazon, or the UPS Flight Forward™ programs. Alternatively, the individual may have obtained an INR test kit, via alternative means (e.g., purchasing the kit at a store, pharmacy, or medical office). The individual collects a blood sample using the INR kit and a drone delivers it to a testing center. Degradation can occur quickly in samples, so the time between sample collection and examining should be minimized. Due to flight limitations of drone technology and sensitivity of the sample, the examination center may be located within 100 miles of the individual and referred to as a regional examination center. The drone may deliver the sample to a temporary holding facility where it is further delivered to an examination center, optionally via non-drone means. The drone may include means for controlling the temperature of the collection device to maintain sample integrity during transit.

The results are then securely communicated to the individual, and optionally any medical staff as requested by the individual. The results may be communicated via email, text message, phone call, or other means, physical or electronic. For example, the results may be transmitted to the individual's smart phone or a medical provider's server. Minimizing the turn-around time is paramount. Utilizing state of the art technology, in embodiments, the methods described herein provide INR test results in less than 24 hours from receipt of the sample at the examination center. As the INR typically needs to be determined on a weekly or bi-weekly basis for patients undergoing anticoagulant therapy, a drone test kit delivery schedule can be established for regular at-home INR testing. In embodiments, the individual receives alerts or is prompted to obtain another INR test.

### Example 7

### Field-Based Forensic Analysis

Field-based forensic tests commonly provide information on the presence and identity of biological stains and body fluids such as blood, saliva, and semen left behind at a crime scene (Morrison J et al. Forensic Sci. Int. 2018; 285: 147-160). Popular tests for blood include the luminol chemiluminescence reaction, commercially available in the ^{BlueStar}® formulation. Several immunoassays are also available for forensic use, such as rapid stain identification (RSID™) kits, which have been developed for the rapid on-site detection of blood, saliva, and semen. Forensic DNA profiling is also routinely performed and traditionally relies on short tandem repeat (STR) loci analysis, although lab-based NGS profiling is being introduced into practice.

A forensics specialist or law enforcement officer is interested in obtaining DNA/RNA sequence analysis from a blood sample at a crime scene. From the field, they access a website or use an application on their smart device to request a test kit to be delivered to the crime scene. The test kit is delivered via an unmanned aerial vehicle (UAV). Alternatively, the forensics specialist or law enforcement officer has a collection device and requests a UAV to come and retrieve the collection device. The individual collects the sample using the test kit (e.g., a swab) and the drone delivers it to an examination center. Degradation can occur quickly in samples, so the time between sample collection and examining should be minimized. Ideally, the sample is placed in a transportation and preservation solution, such as UTM™ or CyMol™ (Copan), to maintain nucleic acid integrity and inactivate pathogen infectivity. Pathogens can still be recovered from refrigerated samples up to 7 days after collection, although the yield typically decreases after the first 2 days. Due to flight limitations of drone technology and sensitivity of the sample, the examination center may be located within 100 miles of the individual and referred to as a regional examination center. The drone may deliver the sample to a temporary holding facility where it is further delivered to an examination center, optionally via non-drone means.

The results are then securely communicated to the individual, and optionally any law enforcement staff as requested by the individual. The results may be communicated via email, text message, phone call, or other means, physical or electronic. Minimizing the turn-around time from test request to results is paramount. Utilizing state of the art sequencers and technology, in embodiments, the methods described herein provide sequencing results in less than 24 hours from receipt of the sample at the examination center. This has considerable potential to enable real-time analysis of evidence at a crime scene before it is subjected to contaminants or tampering.

### P-Embodiments

The present disclosure provides the following illustrative embodiments.

Embodiment P1. A method of detecting a disease in a subject, the method comprising: i) providing a collection device to a subject using an unmanned autonomous vehicle, ii) obtaining a sample from the subject and depositing the sample in the collection device; iii) delivering the collection device to an examination center; iv) identifying whether a disease is present in the sample by examining the sample, and v) detecting a disease in a subject when the presence of a disease is identified in the sample.

Embodiment P2. A method of diagnosing a subject with a disease, the method comprising: i) providing a collection device to a subject using an unmanned autonomous vehicle; ii) obtaining a sample from the subject and depositing the sample in the collection device; iii) delivering the collection device to an examination center; iv) identifying whether a disease is present in the sample by examining the sample, and v) diagnosing a subject with a disease when the presence of a disease is identified in the sample.

Embodiment P3. The method of Embodiment P1 or Embodiment P2, wherein the disease is an infectious disease, an autoimmune disease, hereditary disease, or cancer.

Embodiment P4. A method of detecting a pathogenic organism in a subject, the method comprising: i)providing a collection device to a subject using an unmanned autonomous vehicle; ii) obtaining a sample from the subject and depositing the sample in the collection device; iii) delivering the collection device to an examination center; iv) identifying whether a pathogenic organism is present in the sample by examining the sample, and v) detecting a pathogenic organism in a subject when the presence of a pathogenic organism is identified in the sample.

Embodiment P5. A method of detecting a disease in a subject, the method comprising: i) requesting transportation of an unmanned autonomous vehicle to a destination; ii) obtaining a sample from a subject and depositing the sample in a collection device; iii) providing the collection device to the unmanned autonomous vehicle; iv) delivering the collection device to an examination center; v) identifying whether a disease is present in the sample by examining the sample, and vi) detecting a disease in a subject when the presence of a disease is identified in the sample.

Embodiment P6. The method of any one of Embodiment P1 to Embodiment P5, wherein providing the collection device to a subject is responsive to a request from the subject, a physician, or other health care professional.

Embodiment P7. The method of any one of Embodiment P1 to Embodiment P5, prior to providing a collection device, the method further comprises a subject, a physician, or other health care professional requesting delivery of a collection through a graphical user interface.

Embodiment P8. The method of Embodiment P7, wherein the graphical user interface is a website or an application for use on a mobile computing device.

Embodiment P9. The method of any one of Embodiment P1 to Embodiment P5, prior to providing a collection device, the method further comprises a subject, a physician, or other health care professional requesting delivery of a collection device using a computing device.

Embodiment P10. The method of Embodiment P9, wherein the computing device is a mobile computing device.

Embodiment P11. The method of any one of Embodiment P1 to Embodiment P10, further comprising authenticating the identity of the subject.

Embodiment P12. A method of evaluating a biological sample collected from a subject, the method comprising: i) requesting delivery of a collection device; ii) providing the collection device to the subject using an unmanned autonomous vehicle; iii) obtaining a biological sample from the subject and depositing the biological sample in the collection device; iv) delivering the collection device to an examination center; v) evaluating the biological sample by examining the biological sample.

Embodiment P13. A method of evaluating a biological sample collected from a subject, the method comprising: i) requesting transportation of an unmanned autonomous vehicle to a destination; ii) obtaining a sample from a subject and depositing the sample in a collection device; iii) providing the collection device to the unmanned autonomous vehicle; iv) delivering the collection device to an examination center; v) evaluating the biological sample by examining the biological sample.

Embodiment P14. The method of Embodiment P12 or Embodiment P13, wherein the request for delivery comprises a request through a graphical user interface.

Embodiment P15. The method of Embodiment P12 or Embodiment P13, wherein the request for delivery comprises a request using a computing device.

Embodiment P16. The method of any one of Embodiment P1 to Embodiment P15, wherein examining comprises detecting a biomarker in the sample.

Embodiment P17. The method of any one of Embodiment P1 to Embodiment P15, wherein examining comprises performing an immunoassay, nucleic acid assay, receptor-based assay, nucleic acid sequencing, cytometry, a colorimetric assay, an enzymatic assay, a spectroscopic assay, electrophoresis, an agglutination assay, chromatography, coagulation assay, an electrochemical assay, histological method, a nephelometric assay, a turbidimetric assay, a radioisotope assay, viscometric assay, a protein synthesis assay, culture assay, osmolality assay, or a combination of any of the foregoing.

Embodiment P18. The method of any one of Embodiment P1 to Embodiment P15, wherein examining comprises performing an immunoassay selected from the group radioimmunoassay, counting immunoassay, enzyme immunoassay, enzyme-linked immunosorbent assays, fluoroimmnoassay, electrochemiluminescenceimmunoassay, or chemiluminescenceimmunoassay.

Embodiment P19. The method of any one of Embodiment P1 to Embodiment P15, wherein the examining comprises performing a Clinical Laboratory Improvement Amendments (CLIA)-waived test.

Embodiment P20. The method of any one of Embodiment P1 to Embodiment P15, wherein examining the sample comprises culturing at least a portion of the sample with a microorganism growth medium and a metabolic indicator, wherein the metabolic indicator is capable of being metabolized to produce a metabolic product; and detecting the metabolic product.

Embodiment P21. The method of Embodiment P20, wherein the metabolic indicator comprises resazurin, 5-cyano-2,3-ditolyl tetrazolium chloride (CTC), carboxyfluorescein diacetate succinimidyl ester (CFDA-SE), or luciferin.

Embodiment P22. The method of any one of Embodiment P1 to Embodiment P15, wherein examining the sample comprises performing a nucleic acid assay, wherein the nucleic acid assay comprises contacting at least one primer complementary to a portion of a nucleic acid within the sample.

Embodiment P23. The method of any one of Embodiment P1 to Embodiment P15, wherein examining the sample comprises detecting the presence of antibodies to the pathogenic organism.

Embodiment P24. The method of any one of Embodiment P1 to Embodiment P15, wherein examining the sample comprises sequencing a nucleic acid sequence, or complement thereof, in the sample.

Embodiment P25. The method of any one of Embodiment P1 to Embodiment P24, wherein delivering the collection device to an examination center is performed using an unmanned autonomous vehicle.

Embodiment P26. The method of any one of Embodiment P1 to Embodiment P24, wherein delivering the collection device to an examination center is performed using the same unmanned autonomous vehicle that provided the collection device.

Embodiment P27. The method of any one of Embodiment P1 to Embodiment P24, wherein the examining center is a laboratory.

Embodiment P28. The method of any one of Embodiment P1 to Embodiment P24, wherein the examining center is a Clinical Laboratory Improvement Amendments (CLIA)-compliant laboratory or a CLIA-certified laboratory.

Embodiment P29. The method of any one of Embodiment P1 to Embodiment P28, wherein the sample comprises blood, serum, saliva, urine, tears, gastric and/or digestive fluid, stool, mucus, sputum, sweat, earwax, oil, glandular secretion, semen, or vaginal fluid.

Embodiment P30. The method of any one of Embodiment P1 to Embodiment P28, wherein the sample comprises a nasopharyngeal swab sample, a nasal aspirate sample, or a nasal wash sample.

Embodiment P31. The method of any one of Embodiment P1 to Embodiment P28, wherein the sample comprises bronchial aspirate, endotracheal aspirate, or tracheal aspirate.

Embodiment P32. The method of any one of Embodiment P1 to Embodiment P28, wherein the sample comprises dermal interstitial fluid.

Embodiment P33. The method of any one of Embodiment P1 to Embodiment P32, wherein the collection device comprises a microfluidic paper-based analytical device (gAD), cotton swab, transdermal patch, or device configured to collect and store a bodily fluid.

Embodiment P34. The method of any one of Embodiment P1 to Embodiment P32, wherein the collection device comprises a nasal swab, nasopharyngeal swab, oropharyngeal swab, throat swab, buccal swab, oral fluid swab, stool swab, tonsil swab, vaginal swab, cervical swab, blood swab, or wound swab.

Embodiment P35. The method of any one of Embodiment P1 to Embodiment P32, wherein the collection device comprises a swab, at least one vessel, a buffer, or preservative.

Embodiment P36. The method of any one of Embodiment P1 to Embodiment P32, wherein the collection device comprises a container with integrated thermal control unit or material that provides active or passive cooling.

Embodiment P37. The method of any one of Embodiment P1 to Embodiment P32, wherein the collection device comprises an identifiable tag.

Embodiment P38. The method of Embodiment P37, wherein the identifiable tag is a label, an RFID, or an EEPROM.

Embodiment P39. The method of any one of Embodiment P1 to Embodiment P38, further comprising providing a report to the subject, a healthy agency, a physician, or other health care professional, wherein the report comprises the results from examining the sample.

Embodiment P40. The method of Embodiment P39, wherein the report is provided in less than 24 hours from depositing the sample in the collection device.

Embodiment P41. The method of any one of Embodiment P1 to Embodiment P38, further comprising transmitting data to a computing device or web server, wherein the data comprises data associated with examining the sample.

Embodiment P42. The method of Embodiment P41, wherein the data is transmitted in less than 24 hours from depositing the sample in the collection device.

Embodiment P43. A method of providing a collection device to a subject, the method comprising: i) receiving from a subject a request for delivery of one or more collection devices; ii) providing one or more collection devices to the subject using an unmanned autonomous vehicle, wherein the one or more collection devices include a biological collection device.

Embodiment P44. The method of any one of Embodiment P1 to Embodiment P43, wherein the unmanned autonomous vehicle is an unmanned aerial vehicle.

Embodiment P45. A kit comprising a collection device.

Embodiment P46. The kit of Embodiment P45, wherein the kit is provided to a subject using an unmanned autonomous vehicle.

Embodiment P47. The kit of Embodiment P45, wherein the kit comprises instructions for sample collection.

Embodiment P48. A computing device configured to enable the evaluation of a biological sample collected from a subject, comprising: a processor; memory in electronic communication with the processor; and instructions stored in the memory, the instructions being executable by the processor to: a. request for delivery of a collection device; b. communicate with a server to provide the collection device using an unmanned autonomous vehicle; c. communicate with a server to deliver the collection device containing a biological sample to an examination center; d. receive data associated with the evaluation of the biological sample from the examination center.

Embodiment P49. A system, comprising: at least one data processor; and at least one memory storing instructions, which when executed by the at least one data processor, result in operations comprising: receiving, from a client device, a request for a collection device; responding to the request by at least identifying a first unmanned autonomous vehicle for delivering the collection device, the first unmanned autonomous vehicle being identified based at least on a first location associated with the request and/or a second location of the first unmanned autonomous vehicle; sending, to the first unmanned autonomous vehicle, a first command to deliver the collection device to the first location; and sending, to the first unmanned autonomous vehicle or a second unmanned autonomous vehicle, a second command to deliver the collection device including a sample from the first location to an examination center.

Embodiment P50. The system of Embodiment P49, further comprising an operation to verify the identity of a client, the operation comprising receiving, from a client device, authenticating information and comparing the authenticating information to pre-collected authenticating information, wherein a match between the authenticating information and said pre-collected authenticating information verifies the identity of the client.

Embodiment P51. The system of Embodiment P49, wherein the first location is determined based on a location of the first client device.

Embodiment P52. The system of Embodiment P49, wherein the first location is determined based on a location provided as part of the request.

Embodiment P53. The system of Embodiment P49, wherein the first unmanned autonomous vehicle is identified from a plurality of unmanned autonomous vehicles as being configured for the collection device.

Embodiment P54. The system of Embodiment P49, wherein the first unmanned autonomous vehicle is identified from a plurality of unmanned autonomous vehicles as being in a location that is the closest to the first location.

Embodiment P55. The system of Embodiment P49, wherein the first unmanned autonomous vehicle is identified from a plurality of unmanned autonomous vehicles as having the capacity to receive the collection device.

Embodiment P56. The system of Embodiment P49, wherein the second command is in response to loading of the collection device.

Embodiment P57. The system of Embodiment P49, wherein further comprising an operation for tracking the location of the first unmanned autonomous vehicle, the second unmanned autonomous vehicle, and/or the collection device.

Embodiment P58. An unmanned autonomous vehicle, comprising: at least one data processor; and at least one memory storing instructions, which when executed by the at least one data processor, result in operations comprising: sending a first message including a current location of the unmanned autonomous vehicle and a current capacity of the unmanned autonomous vehicle; receiving a command to deliver a collection device to a location or to deliver the collection device from the location to an examination center; and in response to detecting a deposit and/or a removal of the collection device at the location, sending a second message including an updated location of the unmanned autonomous vehicle and/or an updated capacity of the unmanned autonomous vehicle.

Embodiment P59. The unmanned autonomous vehicle of Embodiment P58, wherein the memory further stores a device ID corresponding to the identity of the unmanned autonomous vehicle and activity log.

Embodiment P60. A system, comprising: an unmanned autonomous vehicles; and a central server communicatively coupled with the unmanned autonomous vehicle, the central server including at least one data processor and at least one memory, the at least one memory may store instructions, which when executed by the at least one data processor, result in operations comprising: receiving, from a client device, a request for a collection device; receiving, from the unmanned autonomous vehicle, a message including a current location of the unmanned autonomous vehicle; responding to the request by at least identifying the unmanned autonomous vehicle for delivering the collection device, the unmanned autonomous vehicle being identified based at least on a location associated with the request and the current location of the unmanned autonomous vehicle; and in response to identifying the unmanned autonomous vehicle for delivering the collection device, sending, to the unmanned autonomous vehicle, a command to deliver the collection device to the location associated with the request.

### Additional Embodiments

The present disclosure provides the following additional illustrative embodiments.

Embodiment 1. A method of detecting a disease in a subject, the method comprising: i) providing a collection device to a subject using an unmanned autonomous vehicle, ii) obtaining a sample from the subject and depositing the sample in the collection device; iii) delivering the collection device to an examination center; iv) identifying whether a disease is present in the sample by examining the sample, and v) detecting a disease in a subject when the presence of a disease is identified in the sample.

Embodiment 2. A method of diagnosing a subject with a disease, the method comprising: i) providing a collection device to a subject using an unmanned autonomous vehicle; ii) obtaining a sample from the subject and depositing the sample in the collection device; iii) delivering the collection device to an examination center; iv) identifying whether a disease is present in the sample by examining the sample, and v) diagnosing a subject with a disease when the presence of a disease is identified in the sample.

Embodiment 3. The method of Embodiment 1 or 2, wherein the disease is an infectious disease, an autoimmune disease, hereditary disease, or cancer.

Embodiment 4. A method of detecting a pathogenic organism in a subject, the method comprising: i)providing a collection device to a subject using an unmanned autonomous vehicle; ii) obtaining a sample from the subject and depositing the sample in the collection device; iii) delivering the collection device to an examination center; iv) identifying whether a pathogenic organism is present in the sample by examining the sample, and v) detecting a pathogenic organism in a subject when the presence of a pathogenic organism is identified in the sample.

Embodiment 5. A method of detecting a disease in a subject, the method comprising: i) requesting transportation of an unmanned autonomous vehicle to a destination; ii) obtaining a sample from a subject and depositing the sample in a collection device; iii) providing the collection device to the unmanned autonomous vehicle; iv) delivering the collection device to an examination center; v) identifying whether a disease is present in the sample by examining the sample, and vi) detecting a disease in a subject when the presence of a disease is identified in the sample.

Embodiment 6. The method of any one of Embodiments 1 to 5, wherein providing the collection device to a subject is responsive to a request from the subject, a physician, or other health care professional.

Embodiment 7. The method of any one of Embodiments 1 to 5, prior to providing a collection device, the method further comprises a subject, a physician, or other health care professional requesting delivery of a collection through a graphical user interface.

Embodiment 8. The method of Embodiment 7, wherein the graphical user interface is a website or an application for use on a mobile computing device.

Embodiment 9. The method of any one of Embodiments 1 to 5, prior to providing a collection device, the method further comprises a subject, a physician, or other health care professional requesting delivery of a collection device using a computing device.

Embodiment 10. The method of Embodiment 9, wherein the computing device is a mobile computing device.

Embodiment 11. The method of any one of Embodiments 1 to 10, further comprising authenticating the identity of the subject.

Embodiment 12. A method of evaluating a biological sample collected from a subject, the method comprising: i) requesting delivery of a collection device; ii) providing the collection device to the subject using an unmanned autonomous vehicle; iii) obtaining a biological sample from the subject and depositing the biological sample in the collection device; iv) delivering the collection device to an examination center; v) evaluating the biological sample by examining the biological sample.

Embodiment 13. A method of evaluating a biological sample collected from a subject, the method comprising: i) requesting transportation of an unmanned autonomous vehicle to a destination; ii) obtaining a sample from a subject and depositing the sample in a collection device; iii) providing the collection device to the unmanned autonomous vehicle; iv) delivering the collection device to an examination center; v) evaluating the biological sample by examining the biological sample.

Embodiment 14. The method of Embodiment 12 or 13, wherein the request for delivery comprises a request through a graphical user interface.

Embodiment 15. The method of Embodiment 12 or 13, wherein the request for delivery comprises a request using a computing device.

Embodiment 16. The method of any one of Embodiments 1 to 15, wherein examining comprises detecting a biomarker in the sample.

Embodiment 17. The method of any one of Embodiments 1 to 15, wherein examining comprises performing an immunoassay, nucleic acid assay, receptor-based assay, nucleic acid sequencing, cytometry, a colorimetric assay, an enzymatic assay, a spectroscopic assay, electrophoresis, an agglutination assay, chromatography, coagulation assay, an electrochemical assay, histological method, a nephelometric assay, a turbidimetric assay, a radioisotope assay, viscometric assay, a protein synthesis assay, culture assay, osmolality assay, or a combination of any of the foregoing.

Embodiment 18. The method of any one of Embodiments 1 to 15, wherein examining comprises performing an immunoassay selected from the group radioimmunoassay, counting immunoassay, enzyme immunoassay, enzyme-linked immunosorbent assays, fluoroimmnoassay, electrochemiluminescenceimmunoassay, or chemiluminescenceimmunoassay.

Embodiment 19. The method of any one of Embodiments 1 to 15, herein the examining comprises performing a Clinical Laboratory Improvement Amendments (CLIA)-waived test.

Embodiment 20. The method of any one of Embodiments 1 to 15, wherein examining the sample comprises culturing at least a portion of the sample with a microorganism growth medium and a metabolic indicator, wherein the metabolic indicator is capable of being metabolized to produce a metabolic product; and detecting the metabolic product.

Embodiment 21. The method of Embodiment 20, wherein the metabolic indicator comprises resazurin, 5-cyano-2,3-ditolyl tetrazolium chloride (CTC), carboxyfluorescein diacetate succinimidyl ester (CFDA-SE), or luciferin.

Embodiment 22. The method of any one of Embodiments 1 to 15, wherein examining the sample comprises performing a nucleic acid assay, wherein the nucleic acid assay comprises contacting at least one primer complementary to a portion of a nucleic acid within the sample.

Embodiment 23. The method of any one of Embodiments 1 to 15, wherein examining the sample comprises detecting the presence of antibodies to the pathogenic organism.

Embodiment 24. The method of any one of Embodiment 1 to 15, wherein examining the sample comprises sequencing a nucleic acid sequence, or complement thereof, in the sample.

Embodiment 25. The method of any one of Embodiments 1 to 24, wherein delivering the collection device to an examination center is performed using an unmanned autonomous vehicle.

Embodiment 26. The method of any one of Embodiments 1 to 24, wherein delivering the collection device to an examination center is performed using the same unmanned autonomous vehicle that provided the collection device.

Embodiment 27. The method of any one of Embodiments 1 to 24, wherein the examining center is a laboratory.

Embodiment 28. The method of any one of Embodiments 1 to 24, wherein the examining center is a Clinical Laboratory Improvement Amendments (CLIA)-compliant laboratory or a CLIA-certified laboratory.

Embodiment 29. The method of any one of Embodiments 1 to 28, wherein the sample comprises blood, serum, saliva, urine, tears, gastric and/or digestive fluid, stool, mucus, sputum, sweat, earwax, oil, glandular secretion, semen, or vaginal fluid.

Embodiment 30. The method of any one of Embodiments 1 to 28, wherein the sample comprises a nasopharyngeal swab sample, a nasal aspirate sample, or a nasal wash sample.

Embodiment 31. The method of any one of Embodiments 1 to 28, wherein the sample comprises bronchial aspirate, endotracheal aspirate, or tracheal aspirate.

Embodiment 32. The method of any one of Embodiments 1 to 28, wherein the sample comprises dermal interstitial fluid.

Embodiment 33. The method of any one of Embodiments 1 to 32, wherein the collection device comprises a microfluidic paper-based analytical device (gAD), cotton swab, transdermal patch, or device configured to collect and store a bodily fluid.

Embodiment 34. The method of any one of Embodiments 1 to 32, wherein the collection device comprises a nasal swab, nasopharyngeal swab, oropharyngeal swab, throat swab, buccal swab, oral fluid swab, stool swab, tonsil swab, vaginal swab, cervical swab, blood swab, or wound swab.

Embodiment 35. The method of any one of Embodiments 1 to 32, wherein the collection device comprises a swab, at least one vessel, a buffer, or preservative.

Embodiment 36. The method of any one of Embodiments 1 to 32, wherein the collection device comprises a container with integrated thermal control unit or material that provides active or passive cooling.

Embodiment 37. The method of any one of Embodiments 1 to 32, wherein the collection device comprises an identifiable tag.

Embodiment 38. The method of Embodiment 37, wherein the identifiable tag is a label, an RFID, or an EEPROM.

Embodiment 39. The method of any one of Embodiments 1 to 38, further comprising providing a report to the subject, a healthy agency, a physician, or other health care professional, wherein the report comprises the results from examining the sample.

Embodiment 40. The method of Embodiment 39, wherein the report is provided in less than 24 hours from depositing the sample in the collection device.

Embodiment 41. The method of any one of Embodiments 1 to 38, further comprising transmitting data to a computing device or web server, wherein the data comprises data associated with examining the sample.

Embodiment 42. The method of Embodiment 41, wherein the data is transmitted in less than 24 hours from depositing the sample in the collection device.

Embodiment 43. A method of providing a collection device to a subject, the method comprising: i) receiving from a subject a request for delivery of one or more collection devices; ii) providing one or more collection devices to the subject using an unmanned autonomous vehicle, wherein the one or more collection devices include a biological collection device.

Embodiment 44. The method of any one of Embodiments 1 to 43, wherein the unmanned autonomous vehicle is an unmanned aerial vehicle.

Embodiment 45. A kit comprising a collection device.

Embodiment 46. The kit of Embodiment 45, wherein the kit is provided to a subject using an unmanned autonomous vehicle.

Embodiment 47. The kit of Embodiment 45, wherein the kit comprises instructions for sample collection.

Embodiment 48. A computing device configured to enable the evaluation of a biological sample collected from a subject, comprising: a processor; memory in electronic communication with the processor; and instructions stored in the memory, the instructions being executable by the processor to: a. request for delivery of a collection device; b. communicate with a server to provide the collection device using an unmanned autonomous vehicle; c. communicate with a server to deliver the collection device containing a biological sample to an examination center; d. receive data associated with the evaluation of the biological sample from the examination center.

Embodiment 49. A system, comprising: at least one data processor; and at least one memory storing instructions, which when executed by the at least one data processor, result in operations comprising: receiving, from a client device, a request for a collection device; responding to the request by at least identifying a first unmanned autonomous vehicle for delivering the collection device, the first unmanned autonomous vehicle being identified based at least on a first location associated with the request and/or a second location of the first unmanned autonomous vehicle; sending, to the first unmanned autonomous vehicle, a first command to deliver the collection device to the first location; and sending, to the first unmanned autonomous vehicle or a second unmanned autonomous vehicle, a second command to deliver the collection device including a sample from the first location to an examination center.

Embodiment 50. The system of Embodiment 49, further comprising an operation to verify the identity of a client, the operation comprising receiving, from a client device, authenticating information and comparing the authenticating information to pre-collected authenticating information, wherein a match between the authenticating information and said pre-collected authenticating information verifies the identity of the client.

Embodiment 51. The system of Embodiment 49, wherein the first location is determined based on a location of the first client device.

Embodiment 52. The system of Embodiment 49, wherein the first location is determined based on a location provided as part of the request.

Embodiment 53. The system of Embodiment 49, wherein the first unmanned autonomous vehicle is identified from a plurality of unmanned autonomous vehicles as being configured for the collection device.

Embodiment 54. The system of Embodiment 49, wherein the first unmanned autonomous vehicle is identified from a plurality of unmanned autonomous vehicles as being in a location that is the closest to the first location.

Embodiment 55. The system of Embodiment 49, wherein the first unmanned autonomous vehicle is identified from a plurality of unmanned autonomous vehicles as having the capacity to receive the collection device.

Embodiment 56. The system of Embodiment 49, wherein the second command is in response to loading of the collection device.

Embodiment 57. The system of Embodiment 49, wherein further comprising an operation for tracking the location of the first unmanned autonomous vehicle, the second unmanned autonomous vehicle, and/or the collection device.

Embodiment 58. An unmanned autonomous vehicle, comprising: at least one data processor; and at least one memory storing instructions, which when executed by the at least one data processor, result in operations comprising: sending a first message including a current location of the unmanned autonomous vehicle and a current capacity of the unmanned autonomous vehicle; receiving a command to deliver a collection device to a location or to deliver the collection device from the location to an examination center; and in response to detecting a deposit and/or a removal of the collection device at the location, sending a second message including an updated location of the unmanned autonomous vehicle and/or an updated capacity of the unmanned autonomous vehicle.

Embodiment 59. The unmanned autonomous vehicle of Embodiment 58, wherein the memory further stores a device ID corresponding to the identity of the unmanned autonomous vehicle and activity log.

Embodiment 60. A system, comprising: an unmanned autonomous vehicles; and a central server communicatively coupled with the unmanned autonomous vehicle, the central server including at least one data processor and at least one memory, the at least one memory may store instructions, which when executed by the at least one data processor, result in operations comprising: receiving, from a client device, a request for a collection device; receiving, from the unmanned autonomous vehicle, a message including a current location of the unmanned autonomous vehicle; responding to the request by at least identifying the unmanned autonomous vehicle for delivering the collection device, the unmanned autonomous vehicle being identified based at least on a location associated with the request and the current location of the unmanned autonomous vehicle; and in response to identifying the unmanned autonomous vehicle for delivering the collection device, sending, to the unmanned autonomous vehicle, a command to deliver the collection device to the location associated with the request.

Embodiment 61. A method of evaluating a biological sample collected from a subject, the method comprising: i) requesting delivery of a collection device; ii) providing the collection device to the subject using an unmanned autonomous vehicle; iii) obtaining the biological sample from the subject and depositing the biological sample in the collection device; iv) delivering the collection device to an examination center; and v) evaluating the biological sample by examining the biological sample.

Embodiment 62. The method of Embodiment 61, further comprising identifying whether a disease is present in the biological sample by examining the sample.

Embodiment 63. The method of Embodiment 61 or 62, further comprising detecting a disease in a subject when the presence of a disease is identified in the biological sample.

Embodiment 64. The method of Embodiment 62 or 63, wherein the disease is an infectious disease, an autoimmune disease, hereditary disease, or cancer.

Embodiment 65. The method of any one of Embodiments 61 to 64, wherein providing the collection device to a subject is responsive to a request for delivery from the subject, a physician, or other health care professional.

Embodiment 66. The method of any one of Embodiments 61 to 65, further comprising authenticating the identity of the subject.

Embodiment 67. The method of any one of Embodiments 61 to 66, wherein examining comprises detecting a biomarker in the sample.

Embodiment 68. The method of any one of Embodiments 61 to 66, wherein examining comprises performing an immunoassay selected from the group radioimmunoassay, counting immunoassay, enzyme immunoassay, enzyme-linked immunosorbent assays, fluoroimmnoassay, electrochemilluminescenceimmunoassay, or chemilluminescenceimmunoassay, or a nucleic acid assay, receptor-based assay, nucleic acid sequencing, cytometry, a colorimetric assay, an enzymatic assay, a spectroscopic assay, electrophoresis, an agglutination assay, chromatography, coagulation assay, an electrochemical assay, histological method, a nephelometric assay, a turbidimetric assay, a radioisotope assay, viscometric assay, a protein synthesis assay, culture assay, osmolality assay, or a combination of any of the foregoing.

Embodiment 69. The method of any one of Embodiments 61 to 66, wherein examining the sample comprises culturing at least a portion of the sample with a microorganism growth medium and a metabolic indicator, wherein the metabolic indicator is capable of being metabolized to produce a metabolic product; and detecting the metabolic product.

Embodiment 70. The method of any one of Embodiments 61 to 66, wherein examining the sample comprises performing a nucleic acid assay, wherein the nucleic acid assay comprises contacting at least one primer complementary to a portion of a nucleic acid within the sample.

Embodiment 71. The method of any one of Embodiments 61 to 70, wherein delivering the collection device to an examination center is performed using an unmanned autonomous vehicle.

Embodiment 72. The method of any one of Embodiments 61 to 70, wherein delivering the collection device to an examination center is performed using the same unmanned autonomous vehicle that provided the collection device.

Embodiment 73. The method of any one of Embodiments 61 to 72, wherein the examining center is a laboratory.

Embodiment 74. The method of any one of Embodiments 61 to 73, wherein the sample comprises blood, serum, saliva, urine, tears, gastric and/or digestive fluid, stool, mucus, sputum, sweat, earwax, oil, glandular secretion, semen, or vaginal fluid.

Embodiment 75. The method of any one of Embodiments 61 to 74, wherein the collection device comprises a microfluidic paper-based analytical device (μPAD), cotton swab, a nasal swab, nasopharyngeal swab, oropharyngeal swab, throat swab, buccal swab, oral fluid swab, stool swab, tonsil swab, vaginal swab, cervical swab, blood swab, wound swab, transdermal patch, or device configured to collect and store a bodily fluid.

Embodiment 76. The method of any one of Embodiments 61 to 74, wherein the collection device comprises a swab, at least one vessel, a buffer, preservative, or an identifiable tag, wherein the identifiable tag is a label, an RFID, or an EEPROM.

Embodiment 77. The method of any one Embodiments 61 to 74, wherein the collection device comprises a container with integrated thermal control unit or material that provides active or passive cooling.

Embodiment 78. The method of any one of Embodiments 61 to 77, wherein the collection device further comprises a self-sterilization modality, wherein the self-sterilization modality is contacted with the sample upon a travel disruption of the unmanned autonomous vehicle.

Embodiment 79. The method of Embodiment 78, wherein the self-sterilization modality comprises heat, an acid, a base, UV light, radiation, a DNAse, a RNAse, or a combination thereof.

Embodiment 80. The method of any one of Embodiments 61 to 79, further comprising providing a report to the subject, a healthy agency, a physician, or other health care professional, wherein the report comprises the results from examining the sample.

Embodiment 81. The method of Embodiment 80, wherein the report is provided in less than 24 hours from depositing the sample in the collection device.

Embodiment 82. The method of any one of Embodiments 61 to 81, further comprising transmitting data to a computing device or web server, wherein the data comprises data associated with examining the sample.

Embodiment 83. The method of any one of Embodiments 61 to 82, wherein the unmanned autonomous vehicle is an unmanned aerial vehicle.

Embodiment 84. A system, comprising: at least one data processor; and at least one memory storing instructions, which when executed by the at least one data processor, result in operations comprising: receiving, from a client device, a request for a collection device; responding to the request by at least identifying a first unmanned autonomous vehicle for delivering the collection device, the first unmanned autonomous vehicle being identified based at least on a first location associated with the request and/or a second location of the first unmanned autonomous vehicle; sending, to the first unmanned autonomous vehicle, a first command to deliver the collection device to the first location; and sending, to the first unmanned autonomous vehicle or a second unmanned autonomous vehicle, a second command to deliver the collection device including a sample from the first location to an examination center.

Embodiment 85. The system of Embodiment 84, further comprising an operation to verify the identity of a client, the operation comprising receiving, from a client device, authenticating information and comparing the authenticating information to pre-collected authenticating information, wherein a match between the authenticating information and said pre-collected authenticating information verifies the identity of the client.

Embodiment 86. The system of Embodiment 84 or 85, wherein the first location is determined based on a location of the first client device or a location provided as part of the request.

Embodiment 87. The system of any one of Embodiments 84 to 86, wherein the first unmanned autonomous vehicle is identified from a plurality of unmanned autonomous vehicles as being configured for the collection device, being in a location that is the closest to the first location, or having the capacity to receive the collection device.

Embodiment 88. The system of any one of Embodiments 84 to 87, wherein the second command is in response to loading of the collection device.

Embodiment 89. The system of any one of Embodiments 84 to 88, wherein further comprising an operation for tracking the location of the first unmanned autonomous vehicle, the second unmanned autonomous vehicle, and/or the collection device.

Embodiment 90. A system, comprising: an unmanned autonomous vehicles; and a central server communicatively coupled with the unmanned autonomous vehicle, the central server comprising at least one data processor and at least one memory, the at least one memory may store instructions, which when executed by the at least one data processor, result in operations comprising: receiving, from a client device, a request for a collection device; receiving, from the unmanned autonomous vehicle, a message including a current location of the unmanned autonomous vehicle; responding to the request by at least identifying the unmanned autonomous vehicle for delivering the collection device, the unmanned autonomous vehicle being identified based at least on a location associated with the request and the current location of the unmanned autonomous vehicle; and in response to identifying the unmanned autonomous vehicle for delivering the collection device, sending, to the unmanned autonomous vehicle, a command to deliver the collection device to the location associated with the request.

What is claimed is:

1. A method of evaluating a biological sample collected from a subject, the method comprising:
i) requesting delivery of a collection device;
ii) providing the collection device to the subject using an unmanned autonomous vehicle;
iii) obtaining the biological sample from the subject and depositing the biological sample in the collection device;
iv) delivering the collection device to an examination center; and
v) evaluating the biological sample by examining the biological sample.

2. The method of claim 1, further comprising identifying whether a disease is present in the biological sample by examining the sample.

3. The method of claim 2, further comprising detecting a disease in a subject when the presence of a disease is identified in the biological sample.

4. The method of claim 2, wherein the disease is an infectious disease, an autoimmune disease, hereditary disease, or cancer.

5. The method of claim 1, wherein providing the collection device to a subject is responsive to a request for delivery from the subject, a physician, or other health care professional.

6. The method of claim 1, further comprising authenticating the identity of the subject.

7. The method of claim 1, wherein examining comprises detecting a biomarker in the sample.

8. The method of claim 1, wherein examining comprises performing an immunoassay selected from the group radioimmunoassay, counting immunoassay, enzyme immunoassay, enzyme-linked immunosorbent assays, fluoroimmnoassay, electrochemilluminescenceimmunoassay, or chemilluminescenceimmunoassay, or a nucleic acid assay, receptor-based assay, nucleic acid sequencing, cytometry, a colorimetric assay, an enzymatic assay, a spectroscopic assay, electrophoresis, an agglutination assay, chromatography, coagulation assay, an electrochemical assay, histological method, a nephelometric assay, a turbidimetric assay, a radioisotope assay, viscometric assay, a protein synthesis assay, culture assay, osmolality assay, or a combination of any of the foregoing.

9. The method of claim 1, wherein examining the sample comprises culturing at least a portion of the sample with a microorganism growth medium and a metabolic indicator, wherein the metabolic indicator is capable of being metabolized to produce a metabolic product;
and detecting the metabolic product.

10. The method of claim 1, wherein examining the sample comprises performing a nucleic acid assay, wherein the nucleic acid assay comprises contacting at least one primer complementary to a portion of a nucleic acid within the sample.

11. The method of claim 1, wherein delivering the collection device to an examination center is performed using an unmanned autonomous vehicle.

12. The method of claim 1, wherein delivering the collection device to an examination center is performed using the same unmanned autonomous vehicle that provided the collection device.

13. The method of claim 1, wherein the examining center is a laboratory.

14. The method of claim 1, wherein the sample comprises blood, serum, saliva, urine, tears, gastric and/or digestive fluid, stool, mucus, sputum, sweat, earwax, oil, glandular secretion, semen, or vaginal fluid.

15. The method of claim 1, wherein the collection device comprises a microfluidic paper-based analytical device (μPAD), cotton swab, a nasal swab, nasopharyngeal swab, oropharyngeal swab, throat swab, buccal swab, oral fluid swab, stool swab, tonsil swab, vaginal swab, cervical swab, blood swab, wound swab, transdermal patch, or device configured to collect and store a bodily fluid.

16. The method of claim 1, wherein the collection device comprises a swab, at least one vessel, a buffer, preservative, or an identifiable tag, wherein the identifiable tag is a label, an RFID, or an EEPROM.

17. The method of claim 1, wherein the collection device comprises a container with integrated thermal control unit or material that provides active or passive cooling.

18. The method of claim 1, wherein the collection device further comprises a self-sterilization modality, wherein the self-sterilization modality is contacted with the sample upon a travel disruption of the unmanned autonomous vehicle.

19. The method of claim 18, wherein the self-sterilization modality comprises heat, an acid, a base, UV light, radiation, a DNAse, a RNAse, or a combination thereof.

20. The method of claim 1, further comprising providing a report to the subject, a healthy agency, a physician, or other health care professional, wherein the report comprises the results from examining the sample.

21. The method of claim 20, wherein the report is provided in less than 24 hours from depositing the sample in the collection device.

22. The method of claim 1, further comprising transmitting data to a computing device or web server, wherein the data comprises data associated with examining the sample.

23. The method of claim 1, wherein the unmanned autonomous vehicle is an unmanned aerial vehicle.

24. A system, comprising:
at least one data processor; and
at least one memory storing instructions, which when executed by the at least one data processor, result in operations comprising:
receiving, from a client device, a request for a collection device;
responding to the request by at least identifying a first unmanned autonomous vehicle for delivering the collection device, the first unmanned autonomous vehicle being identified based at least on a first location associated with the request and/or a second location of the first unmanned autonomous vehicle;
sending, to the first unmanned autonomous vehicle, a first command to deliver the collection device to the first location; and
sending, to the first unmanned autonomous vehicle or a second unmanned autonomous vehicle, a second command to deliver the collection device including a sample from the first location to an examination center.

25. The system of claim 24, further comprising an operation to verify the identity of a client, the operation comprising receiving, from a client device, authenticating information and comparing the authenticating information to pre-collected authenticating information, wherein a match between the authenticating information and said pre-collected authenticating information verifies the identity of the client.

26. The system of claim 24, wherein the first location is determined based on a location of the first client device or a location provided as part of the request.

27. The system of claim 24, wherein the first unmanned autonomous vehicle is identified from a plurality of unmanned autonomous vehicles as being configured for the collection device, being in a location that is the closest to the first location, or having the capacity to receive the collection device.

28. The system of claim 24, wherein the second command is in response to loading of the collection device.

29. The system of claim 24, wherein further comprising an operation for tracking the location of the first unmanned autonomous vehicle, the second unmanned autonomous vehicle, and/or the collection device.

30. A system, comprising:
an unmanned autonomous vehicles; and
a central server communicatively coupled with the unmanned autonomous vehicle, the central server comprising at least one data processor and at least one memory, the at least one memory may store instructions, which when executed by the at least one data processor, result in operations comprising:
receiving, from a client device, a request for a collection device;
receiving, from the unmanned autonomous vehicle, a message including a current location of the unmanned autonomous vehicle;
responding to the request by at least identifying the unmanned autonomous vehicle for delivering the collection device, the unmanned autonomous vehicle being identified based at least on a location associated with the request and the current location of the unmanned autonomous vehicle; and
in response to identifying the unmanned autonomous vehicle for delivering the collection device, sending, to the unmanned autonomous vehicle, a command to deliver the collection device to the location associated with the request.
